(19) United States
(12) Patent Application Publication
(10) Pub. No.: US 2026/0299321 A1
(43) Pub. Date: Oct. 1, 2026

(54) System for Measuring Facial Dimensions

(71) Applicant: Bryant Medical Ltd., Lyne, Chertsey (GB)

(72) Inventors: Neha Raghava, Lyne, Chertsey (GB); Priyam Patel, Lyne, Chertsey (GB); Ibrahim Animashaun, Lyne, Chertsey (GB); Daniel Fiuza Dosil, Lyne, Chertsey (GB); Klauss Andrei Agheorghiesei, Lyne, Chertsey (GB)

(21) Appl. No.: 19/479,666

(22) PCT Filed: Apr. 26, 2024
(86) PCT No.: PCT/GB2024/051123

§ 371 Date: Oct. 28, 2025

(30) Foreign Application Priority Data
Apr. 28, 2023 (GB) 2306395.1

(51) Int. Cl. G02C 13/00; G06T 5/70; G06T 7/50; G06T 7/73; G06V 10/34; G06V 40/16; G06V 40/60

(52) U.S. Cl. CPC G02C 13/005; G06T 5/70; G06T 7/50; G06T 7/73; G06V 10/34; G06V 40/165; G06V 40/171; G06V 40/67; G06T 2207/10016; G06T 2207/10028; G06T 2207/30201

(57) ABSTRACT

A method of measuring facial dimensions is provided, comprising: receiving an image of a user's face; and performing validation of the image.

### FIELD OF THE INVENTION

The present invention relates to a tool for and method of measuring dimensions of a user's face remotely, in particular for the manufacture of bespoke loupes.

### BACKGROUND OF THE INVENTION

Many professionals such as dentists and surgeons use refractive loupes when performing procedures. Loupes are small magnification devices used to see small details more closely. Loupes are worn by dentists not only to increase the details that can be seen, but also to improve posture by avoiding slouching to view inside a mouth. In many instances, dentists mount a headlight, with an associated battery pack, on the loupes to increase visibility. Loupes are also common in other healthcare-related professional sectors such as surgery and (typically single eyeglass loupes) in professional sectors such as jewellery, geology and printing.

Loupes are preferably made bespoke to each user, in order to improve not only comfort but also to ensure the magnifying telescopes are correctly positioned to ensure efficient magnification for the particular user's eyes. The loupes are therefore ‘fitted’ to the dentist's (or other user's) face. Loupes configured for higher magnifications have a smaller field of view and so any margin for error in the location of the apertures relative to a user's pupils is much smaller. For example, if the interpupillary distance (IPD) is within 1 to 2 mm of the correct distance, lower magnification loupes may still perform effectively for the user. However, for higher magnification loupes, this inaccuracy may lead to the telescopes not performing effectively for that user. For higher magnifications, it therefore becomes more important that the telescopes are positioned correctly for each user.

Typically, fitting loupes requires a specially trained professional manually taking measurements of an individual's face in order to ensure an accurate fit. In some instances, measurement of IPD can be performed remotely via analysis of images of a user's face; however, this can lead to inaccurate values. The present invention intends to provide an accurate and efficient tool for and method of obtaining the precise measurements required for fitting loupes.

### SUMMARY OF THE INVENTION

Aspects and embodiments of the present invention are set out in the appended claims. These and other aspects and embodiments of the invention are also described herein.

According to a first aspect of the invention, there is provided a method of measuring facial dimensions and/or measurements, comprising: receiving an image of a user's face; and performing validation of the image.

Validation of the image can advantageously ensure that the image is of sufficient quality for determining facial dimensions and/or measurements. Preferably, this is performed in real-time for live video (simultaneous to the recording of the images), to prevent a user needing to upload multiple images. In such an instance, the method may comprise receiving an image of a user's face and performing real-time validation of the image. Receiving the image may comprise receiving live video images. In some implementations, receiving an image may comprise at least one of and/or any combination of: receiving at least one static image; receiving a series of static images; receiving video images; receiving a series of video frames (for example, as static images); receiving live video images. For example, it may comprise receiving video images and at least one static image, preferably wherein the at least one static image is high-resolution. In some implementations, the validation may be performed offline (i.e. not live) and/or after an interval. This may be in addition to or alternatively to real-time validation. The offline and/or delayed validation may be performed on any image type, and any combination of image types. In some implementations, a combination of real-time validation and offline validation may be used; these may be performed on the same or different images. For example, real-time validation may be performed on live video images, and offline validation performed on high-resolution static images corresponding to ‘snapshots’ of the live video images. A high-resolution image is typically defined as having a resolution of at least 300 pixels per inch (PPI) and/or 1 MPx, while preferably a high-resolution image used in the described method has a resolution of at least 5 MPx, more preferably at least 7 MPx.

Preferably the validation comprises assessing the orientation of the user's face within the image. This can help to ensure the determined dimensions are correct.

The method may further comprise outputting, preferably in real-time, instructions to the user as to how to improve image quality, in dependence on the validation, preferably the real-time validation. The instructions may be audible and/or visible, and verbal and/or graphic and/or symbolic. This can streamline the process of obtaining an image of sufficient quality.

The method may further comprise: outputting instructions to the user as to how to reorient their head in dependence on the validation. The method may further comprise instructing a user how to reorient a camera for capturing the image and/or how to reorient the user's head and the camera relative to one another. This may include outputting instructions to the user as to how to reorient their head relative to the camera, and/or vice versa, in dependence on the validation. The instructions may be audible and/or visible, and verbal and/or graphic and/or symbolic. This can streamline the process of obtaining an image of sufficient quality by ensuring the correct facial dimensions are being determined.

In some implementations, the method may further comprise: capturing an image in dependence on the validation, preferably recording an image or more than one image (i.e. at least one image) in dependence on sufficient image quality, and/or preferably in dependence on the orientation of the user's face. This can ensure only an image of sufficient quality, for example, in which the correct dimensions are being determined, is recorded and/or captured and/or processed.

Preferably, the assessing the orientation of the user's face is based on locations of facial landmarks. Facial landmarks may be points on a face. The facial landmarks may be defined in a library. Preferably, more than 10 facial landmarks, and/or preferably more than 50 facial landmarks, and/or preferably more than 100 landmarks, and/or preferably more than 250 landmarks, and/or preferably more than 500 landmarks.

According to a further aspect of the invention, there is provided a method of assessing the orientation of a user's face within an image wherein the orientation is assessed using locations of facial landmarks.

This can help to ensure that correct facial dimensions are provided within the image (e.g. the user is looking in the correct orientation).

The method may be individually applied to frames of a video, preferably frames of a video in which a user's head moves relative to a camera, more preferably wherein the user's head moves relative to a camera in a routine, and/or preferably wherein the position of the user's head relative to a camera changes in at least one of: roll, pitch and yaw and/or a combination of at least two of: roll, pitch and yaw. This may be achieved by the movement of the head or the camera, or a combination of the two. The movement typically can produce a series of images showing the user's head from different angles. The method can therefore account for different possible orientations of the head away from the correct orientation. The routine may comprise adjusting head tilt and/or yaw and/or pitch and/or roll. This may be done symmetrically (e.g. left then right). The routine may be a sequence.

The method may be applied to a static image or a series of static images, preferably a high-resolution static image or series of high-resolution static images. A high-resolution image is typically defined as having a resolution of at least 300 pixels per inch (PPI) and/or 1 MPx, while preferably a high-resolution image used in the described method has a resolution of at least 5 MPx, more preferably at least 7 MPx.

The method may further comprise determining the facial landmarks using facial mapping techniques. These may be used to locate and/or follow over time facial landmarks.

The method may further comprise determining at least one eye pupil centre location by interpolating the locations of facial landmarks defined around an eye of a user, preferably around a pupil of the eye, and/or around an iris of the eye. Facial mapping and/or facial landmarks may not define pupil centre. This may improve the accuracy of the determination of pupil centre.

According to a further aspect of the invention, there is provided a method of determining eye pupil centre location in an image of a user's face by: determining facial landmarks, preferably using facial mapping techniques; and interpolating locations of facial landmarks defined around an eye, preferably around a pupil of the eye, and/or around an iris of the eye; and preferably further comprising assessing the orientation of the user's face within the images.

This can improve the accuracy of the determination of pupil centre, which is typically not located in facial mapping. This, in turn, can improve the accuracy of calculation of interpupillary distance (IPD).

The interpolating may comprise determining a central point of a polygon formed by the facial landmarks, preferably wherein the facial landmarks form the vertices and/or wherein the polygon is preferably formed by the nearest facial landmarks (e.g. the nearest facial landmarks to the eye and/or pupil of the eye, and/or around iris of the eye), and/or preferably wherein the polygon is a quadrilateral. The polygon may be two-dimensional (2D) or three-dimensional (3D). The polygon may also have a number of sides different to four (i.e. it may be a shape other than a quadrilateral).

The method may further comprise determining the eye pupil centre location for a first eye and a second eye, and determining the interpupillary distance as the distance between the eye pupil centre location of the first eye and the eye pupil centre location of the second eye.

The interpupillary distance (IPD) can preferably be defined as the distance between a first determined pupil centre and a second determined pupil centre.

The method may further comprise determining the eye pupil centre location for a first eye and a second eye, and determining a relative pupillary height (PH) as the relative height of the eye pupil centre location of the first eye and the eye pupil centre location of the second eye.

The relative pupillary height (PH) can be defined as the relative height of a first determined pupil centre location (i.e. of a first eye) and a second pupil centre (i.e. of a second eye), preferably when the head is correctly orientated.

The method may further comprise determining at least one eye pupil centre location, defining a datum point, and determining the distance between the at least one eye pupil centre location and the datum point, preferably wherein said distance defines a monocular pupillary distance and/or preferably wherein the datum point is located in the vicinity of the nose bridge (i.e. bridge of the nose). The datum point may be the nose bridge and/or a point defined on the nose bridge and/or a reference marker located in the location of the nose bridge (for example, the nose bridge of reference frames). The monocular pupillary distance may be a horizontal component of the line between the at least one eye pupil centre location and the datum point, preferably wherein the image and/or the user's face is correctly oriented.

The method may further comprise using facial landmarks to define at least one of: face length; face width; face perimeter; nose to cheek line; monocular pupillary distance; and interpupillary distance.

The face length may be defined as a line from a top central facial landmark to bottom central facial landmark. The face perimeter may be defined as a line joining/connecting/passing through the outermost facial landmarks. The face width distance may be defined as a line from a central left landmark to a central right landmark.

Preferably, the validation and/or the assessing the orientation of a user's face may comprise determining parameters representative of at least one of: distance from camera, facial roll, facial pitch, and facial yaw; preferably determining a range of parameters encompassing all of: distance from camera, facial roll, facial pitch, and facial yaw. These can account for/consider different possible facial orientations.

Preferably, the nose to cheek line is a line joining a nose landmark to a cheek landmark.

Preferably, the validation and/or assessing the orientation of a user's face comprises determining at least one nose to cheek angle (for example, this may be correlated to facial pitch), wherein the nose to cheek angle is the angle between a nose to cheek line and the horizontal.

Some implementations may comprise determining an average value of nose to cheek angle (e.g. left nose to cheek angle and right nose to cheek angle); and preferably assessing the average value in relation to an acceptable range.

The validation may comprise determining ratio of area of the face to the area of the image, preferably wherein the validation comprises determining a ratio of area within the face perimeter to total area of image. This can aid in determining the user is a correct distance from the camera.

The validation may comprise determining distance from the camera using focal length of the camera and at least one image of at least one known dimension. For example, the known dimension may comprise at least one distance between reference markers and/or an iris diameter. Iris diameters are known to be 11.71 mm. The focal length of the camera may be known, or may be approximated based on common/typical focal lengths of the camera type being used. For example, focal lengths of mobile phone cameras are typically 23-26 mm (with some as high as 32 mm).

The validation process may comprise performing a check of one or more technical and/or computational and/or processing parameters and/or performing a check of image resolution.

The validation and/or the assessing the orientation of a user's face may comprise determining a ratio of a left area of face to a right area of face. This can aid in determining facial yaw. The left area of face may be the area within perimeter to the left of the length line, and right area of face may be the area within perimeter to the right of the length line.

The validation and/or the assessing the orientation of a user's face may comprise determining an angle between length line and the horizonal and/or an angle between width line and the horizontal. This can aid in determining facial roll.

The validation and/or the assessing the orientation of a user's face may comprise determining a ratio of face length to height of image and/or a ratio of face width to width of image and/or a ratio of face length to face width.

The method may comprise comparing determined parameters with respective pre-defined acceptable ranges. The pre-defined acceptable ranges may be manually and/or centrally and/or locally defined. The pre-defined acceptable ranges may be based on analysis of images of faces, preferably images of faces (for example, manually) marked acceptable for obtaining facial measurement. These images of faces may typically show the faces in an acceptable orientation for obtaining facial measurement. These images may have been captured for or during performing manual measurement.

The method may further comprise outputting instructions to a user how to reorient their head and/or a or the camera in dependence on one or more determined parameters being found to be outside their respective acceptable range. This may be output in real time/live (so that a user may be able to reorient their head immediately).

The method may comprise automatically capturing an image in dependence on the validation, preferably in dependence on the assessing the orientation of a user's face and/or preferably in dependence on determining that all the parameters are within their respective acceptable ranges.

The facial landmarks may be defined live, preferably wherein the facial mapping techniques are applied live. This may be such that it follows the user's face in the image.

The method may further comprise smoothing the positions of the facial landmarks, preferably wherein the smoothing comprises determining average positions over time and/or across a series of images, more preferably normalizing positions over time and/or across a series of images.

According to a further aspect of the invention, there is provided a method of measuring facial dimensions, comprising: receiving an image, preferably receiving video images (which, for example, in some implementations may be live) and/or preferably receiving a series of images; defining facial landmarks; and smoothing the positions of the facial landmarks; preferably wherein the smoothing comprises determining average positions over time and/or across a series of images, more preferably normalizing positions over time and/or across a series of images. The series of images may be a series of frames of a video. Preferably the series of images are captured with a short delay between consecutive images, for example no more than 1 second, preferably no more than 0.5 seconds.

The method may further comprise defining an animation of the user's face using the facial landmarks, preferably using the facial mapping, and/or preferably wherein the animation is a face mesh, and/or preferably comprising a facial topology visualization, and/or preferably the animation comprises mesh lines joining the facial landmarks. An ‘animation’ may be any visual representation. Preferably, the animation is output to a display.

The method may further comprise overlaying the animation, preferably in real time, preferably such that it is displayed over the user's face in the image. The animation may preferably move with the user's face in the image (for example, in translation, rotation, expression etc., and/or the landmarks may be tracked).

The method may further comprise displaying a marker, for example outputting a marker to a display. The display may preferably be the same display to which the animation is output. This can be used as a focus point for a user. This can help to keep the focus of the user consistent, and hence the IPD consistent.

The method may further comprise transmitting an image to a system storage (for example, a server), preferably an unedited (for example, raw, unedited etc.) image. This may preferably be a high-resolution image. A high-resolution image is typically defined as having a resolution of at least 300 pixels per inch (PPI) and/or 1 MPx, while preferably a high-resolution image used in the described method has a resolution of at least 5 MPx, more preferably at least 7 MPx.

The method may further comprise performing validation at a first location and performing image calibration at a second location, preferably wherein the first location is a user device and a second location is a system storage (for example, a server).

The algorithm may be coded in Python and/or JavaScript and/or a combination of the two. Preferably, the method may be run on a browser (e.g. a web browser). Preferably, the orientation may be performed via a browser (e.g. a web browser).

According to a further aspect, there is provided a method of measuring facial dimensions, comprising performing validation of an image at a first location and performing calibration of the image at a second location, preferably wherein the first location is a user device and a second location is a system storage.

This can facilitate real time imaging and validation over a web-browser, for example, negating the need to download/use a dedicated application.

The method may further comprise calibrating dimensions of the image to physical dimensions by reference to an object of known physical dimensions in the image. The image dimensions may be pixel dimensions.

The method may further comprise determining an orientation of the object of known physical dimensions in the image by comparing dimensions and/or symmetry of the object in the image to known physical dimensions and/or physical symmetry of the object.

In some implementations, the object of known physical dimensions may be a pair of reference frames. The reference frames may comprise at least one marker and/or identifying feature at a known location, preferably wherein the reference frames comprise a series of markers and/or identifying features at known locations, and/or preferably wherein the reference frames comprise a series of markers and/or identifying features at known spaced intervals. The reference frames may resemble a pair of glasses, and preferably are wearable by a user.

The method may further comprise extracting a segment of the image containing the object, preferably wherein the extracting is unsupervised.

According to a further aspect, there is provided a method of calibrating an image, comprising extracting a segment of the image containing an object of known physical dimensions, preferably wherein the extracting is unsupervised.

This can provide a cleaner image on which to perform calibration and/or validation. The extracting being unsupervised encompasses being automatic, and/or utilizing machine learning, and/or utilizing Al etc.

The method may further comprise calibrating the image by performing depth analysis using at least one of: LIDAR technology; RADAR technology; SONAR technology; Structured Light; and Photogrammetry.

The method may further comprise capturing a series of images from different positions relative to a user's head. The method may further comprise determining point cloud data to determine a 3D mesh, preferably a 3D mesh of a user's face.

The method of any preceding claim, further comprising capturing a series of images from different positions relative to a user's head. The series of images preferably show different the use's head from different angles. These may be captured by moving the camera relative to the user and vice versa, or a combination of both.

The method may further comprise outputting an image overlay comprising virtual reference frames and/or virtual loupes and/or virtual glasses. These may be an animation of frames on a display. These may correspond to reference frames and/or loupes and/or glasses. Preferably, they are displayed in location on the display (i.e. it appears as if the user is wearing the virtual reference frames and/or loupes and/or glasses). Preferably, depth analysis may be used. For example, depth analysis may be used to determine accurate positioning and scaling of the virtual reference frames and/or virtual loupes and/or virtual glasses. Depth analysis may be performed using any of: LIDAR technology; RADAR technology; SONAR technology; Structured Light; and Photogrammetry, or any combination thereof.

The method may further comprise manufacturing bespoke loupes, preferably using the facial dimensions and/or location. (For example, interpupillary distance and/or pupil centre location).

Preferably, (the) facial dimensions comprise at least one of (and preferably at least two of): interpupillary distance (IPD), relative pupillary height, monocular pupillary distance (i.e. distance from a defined datum point to a first pupil centre and/or distance from a defined datum point to a second pupil centre), and vertex. (These may be considered to be the facial dimensions of interest.) The relative pupillary height preferably refers to the height of pupil centres relative to one another, and/or relative to the horizontal.

Preferably, frame size may be determined in dependence on interpupillary distance (IPD).

Preferably, a position of telescopes on lenses is determined in dependence on interpupillary distance (IPD) and relative pupillary height. This can help to ensure the magnification lenses work (efficiently) for the particular user/wearer.

Preferably, the working distance is determined in dependence on the user's height. (And, in some instances, gender.)

According to a further aspect, there is provided a computer program product comprising computer implementable instructions for causing a programmable computer device to carry out the method as described above (and in the description, figures and claims).

The computer implementable instructions may be configured to causing more than one computer device to carry out the method, preferably a local device (e.g. local to a user) and a central device (e.g. a server).

These may be configured to use Python and/or JavaScript and/or a combination of the two. Preferably, these are configured to run in a web browser.

According to a further aspect, there is provided a tool for measuring facial dimensions, wherein the tool is configured for receiving an image of a user's face, and configured to perform validation of the image.

The tool may be configured to receive at least one of, and any combination of: at least one static image; a series of static images; video images; live video images.

The tool may be configured to perform real-time validation of the image or images. The tool may be configured to assess orientation of the user's face within the image. The tool may output instructions how to reorient the user's face and how to improve image quality in dependence on the validation. The tool may further be configured to capture an image in dependence the validation.

The validation and/or the assessing the orientation of a user's face may comprise determining parameters representative of at least one of: distance from camera, facial roll, facial pitch, and facial yaw; preferably determining a range of parameters encompassing all of: distance from camera, facial roll, facial pitch, and facial yaw.

The tool may be configured to receive and validate a series of images of a user's face in which the user's face move relative to the camera in at least one of: facial roll, facial pitch, and facial yaw.

The tool may preferably be configured to determine facial landmarks. For example, this may be performed by facial mapping techniques. The tool may be configured to determine at least one eye pupil centre location by interpolating locations of facial landmarks around an eye of a user, preferably around a pupil of the eye, and/or around an iris of the eye.

The tool is preferably configured to define, preferably using facial landmarks, at least one of: face length; face width; face perimeter; nose to cheek line; monocular pupillary distance; and interpupillary distance. These may be considered to be relevant facial dimensions. The tool is preferably configured to output instructions for manufacturing bespoke loupes in dependence on the facial dimensions. The tool may be configured to determine at least one of, and preferably two of: interpupillary distance; monocular pupillary distance; relative pupillary height, and vertex. A position of telescopes on lenses of the bespoke loupes may be determined in dependence on at least one of, and preferably at least two of: interpupillary distance (IPD), monocular pupillary distance, and relative pupillary height.

According to a further aspect, there is provided a tool for measuring facial dimensions, preferably remotely, for the manufacture of bespoke loupes.

According to a further aspect, there is provided a tool for measuring for bespoke loupes, preferably remotely, configured to determine facial measurements from one or more images of a user's face, preferably configured to perform orientation processing of the one or more images of a user's face. Preferably the tool is for measuring for the manufacture of bespoke loupes.

According to a further aspect, there is provided a tool for measuring facial dimensions, configured to perform orientation processing in real time.

According to a further aspect, there is provided a tool for measuring facial dimensions remotely, configured to run an orientation process locally on a user device, and preferably wherein a calibration process is performed on a central server.

This can help to facilitate the real time processing of images via a web browser, avoiding the need for a dedicated (local) application and/or software.

The tool preferably comprises a computer program product comprising computer implementable instructions for causing a programmable computer device to record and/or capture image or images from a camera. This may be a static camera and/or a video camera. Preferably the camera is located locally (i.e. can be located in many different locations, local to a user). The tool preferably further comprises a computer program product comprising computer implementable instructions for causing a programmable computer device (e.g. the local computer device) to send the image or images to a central programmable computer device and/or server.

Preferably, the tool further comprises a computer program product comprising computer implementable instructions for causing the central programmable computer device and/or server to receive and process the image or images. The processing of the image or images preferably comprises the method as described above. The processing preferably comprises validating the image or images, preferably in dependence on orientation of a user's face. The processing preferably further comprises determining facial dimensions, for example comprising at least one of, preferably at least two of: interpupillary distance (IPD), monocular pupillary distance, and relative pupillary height.

Preferably, the tool further comprises a computer program product comprising computer implementable instructions for causing manufacture of bespoke loupes. The instructions may be sent to and/or implemented in machinery configured to manufacture loupes. Preferably, the manufacture of the bespoke loupes is performed using and/or in dependence on the facial dimensions.

The tool may comprise computer implementable instructions and/or at least one programmable computer device. Preferably, the tool comprises computer implementable instructions and/or at least one local programmable computer device and at least one central programmable computer device. The tool may preferably further comprise machinery for manufacturing bespoke loupes. Preferably the at least one local programmable computer device and at least one central programmable computer device and/or machinery for manufacturing bespoke loupes are configured to communicate with one another (in any combination).

Preferably, a first copy of an or the image or images is processed locally and a second copy is processed centrally (for example, at a server).

Preferably, the tool is configured to output instructions to a user regarding orientation of a user's head. The tool may comprise a user interface to which the instructions are output and/or the tool may comprise computer implementable instructions for causing a programmable computer device to output the instructions, preferably wherein the computer device is a or the local device.

Preferably, the tool is configured to perform the method as described above and in the description and figures, and as recited in the claims.

According to a further aspect, there is provided a system for measuring for bespoke loupes (for example, for manufacture of bespoke loupes), configured to determine facial measurements from one or more images of a user's face, the system comprising: a central server; and a user device comprising (or in connection with) a camera device; wherein images from the camera device are separately processed, both locally at the user device, and at the server.

The server may be the system storage.

Preferably, the user device is configured to display the images, preferably a processed version of the images, more preferably a smoothed version of the images and/or preferably with an animation overlay. The user device may comprise a user interface for displaying the images.

Preferably, the user device is configured to perform orientation processing, preferably including outputting instructions to a user how to reorient their head.

Preferably, the server is configured to perform calibration processing, preferably on an unprocessed version of the images.

The system may comprise the tool as described above, and in the description and figures.

In further aspects of the invention are provided a tool (as above, and in the description and figures) and/or a system (as above, and in the description and figures), configured to perform the method (as above, and in the description and figures).

Furthermore, the invention may comprise any, some of, or all of the features described above and/or following features in any appropriate order or combination: a measurement tool running via a browser (for example, a web browser) (i.e. it does not need a dedicated app); the system calculates/determines the location of user's pupils (e.g. pupil location) using interpolation; an algorithm for checking the correct orientation of the user's head (within an image); an algorithm for performing calibration to determine dimensions (for example physical dimensions); using face mapping software for measurement of facial dimensions; using video (rather than static) images to provide consistent frame size (for example, in pixels); using metadata of images (e.g. video), for example to determine focal length/zoom; performing a calibration workflow; performing a calibration workflow in real time; performing a calibration workflow to ensure correct calibration in one or more orientations and/or positionings (e.g. parameters) of: pitch, yaw, roll and distance from camera; restarting workflow if any of the orientations and/or positionings are unacceptable and/or incorrect and/or unsuitable and/or insufficient; recording an image only in dependence on all the orientations and/or positionings being correct and/or suitable and/or acceptable; recording/capturing of an image comprising recording/capturing/locating a corresponding frame (or frames) of a copy of the image/video (for example, an unprocessed and/or raw and/or unedited version/copy); splitting the image (static or video) to be processed locally and centrally (e.g. at a server); preferably wherein validation and/or orientation processing is performed locally; preferably wherein calibration and/or measurement determination (and/or output) is performed centrally (e.g. at a server); recording a video of a user adjusting facial pitch and/or yaw and/or roll and/or distance, and then performing orientation processing (as described) to determine frames for analysis (for example, calibration analysis and/or determination of measurements; the frames so processed may be corresponding frames in a copy of the video.

The invention extends to methods and/or apparatus substantially as herein described with reference to the accompanying drawings.

Any apparatus feature as described herein may also be provided as a method feature, and vice versa.

Any feature in one aspect of the invention may be applied to other aspects of the invention, in any appropriate combination. In particular, method aspects may be applied to apparatus aspects, and vice versa. Furthermore, any, some and/or all features in one aspect can be applied to any, some and/or all features in any other aspect, in any appropriate combination.

Furthermore, features implemented in hardware may be implemented in software, and vice versa. Any reference to software and hardware features herein should be construed accordingly.

Any apparatus feature as described herein may also be provided as a method feature, and vice versa. As used herein, means plus function features may be expressed alternatively in terms of their corresponding structure, such as a suitably programmed processor and associated memory.

It should also be appreciated that particular combinations of the various features described and defined in any aspects of the disclosure can be implemented and/or supplied and/or used independently.

The disclosure also provides a computer program and a computer program product comprising software code adapted, when executed on a data processing apparatus, to perform any of the methods described herein, including any or all of their component steps.

The disclosure also provides a computer program and a computer program product comprising software code which, when executed on a data processing apparatus, comprises any of the apparatus features described herein.

The disclosure also provides a computer program and a computer program product having an operating system which supports a computer program for carrying out any of the methods described herein and/or for embodying any of the apparatus features described herein.

The disclosure also provides a computer readable medium having stored thereon the computer program as aforesaid.

The disclosure also provides a signal carrying the computer program as aforesaid, and a method of transmitting such a signal.

Where the disclosure references the keyboard being arranged to operate in a certain way, this may comprise the control unit being arranged to operate in a certain way (and vice versa).

As used herein, a touch of the user may refer to a touch of the user using an appendage of the user (e.g. a finger). Equally, a touch of the user may refer to a touch of the user using an implement, such as a stylus.

The disclosure extends to methods and/or apparatus substantially as herein described with reference to the accompanying drawings.

It should also be appreciated that particular combinations of the various features described and defined in any aspects of the invention can be implemented and/or supplied and/or used independently.

The term ‘comprising’ as used in this specification and claims preferably means ‘consisting at least in part of’. When interpreting statements in this specification and claims which include the term ‘comprising’, other features besides the features prefaced by this term in each statement can also be present. Related terms such as ‘comprise’ and ‘comprised’ are to be interpreted in a similar manner.

### BRIEF DESCRIPTION OF THE FIGURES

One or more aspects will now be described, by way of example only and with reference to the accompanying drawings having like-reference numerals, in which:

FIG. 1 shows a schematic overview of an exemplary system for implementing the invention;

FIG. 2 shows a schematic workflow of software processes;

FIG. 3 shows landmark points overlaid on a schematic camera feed showing a user's face;

FIG. 4 shows a schematic view of a camera feed interpolating to find the centre of a pupil of a user's eye;

FIG. 5 shows an exemplary schematic illustration of a browser display;

FIG. 6 shows a schematic illustration of tilt, pitch and yaw with respect to a user's head;

FIG. 7 shows exemplary analysis of landmark points overlaid on a schematic camera feed showing a user's face;

FIG. 8 shows further exemplary analysis of landmark points overlaid on a schematic camera feed showing a user's face;

FIG. 9 shows an exemplary workflow of orientation processes;

FIG. 10a shows a front view of reference frames;

FIG. 10b shows a side view of reference frames;

FIG. 11 shows extraction of a segment from an exemplary schematic view of a camera feed showing a user's face wearing reference frames;

FIG. 12a shows a front view of an exemplary pair of loupes;

FIG. 12b shows a side view of an exemplary pair of loupes;

FIG. 13 shows a flow of user interactions with an example of the measuring tool;

FIG. 14 shows an exemplary schematic illustration of user head movements for an alternative embodiment of the present invention.

### DETAILED DESCRIPTION

The present invention relates to a tool for performing remote measurement of a user's face in order to fit bespoke magnification loupes, typically for medical or dental use.

### System Overview

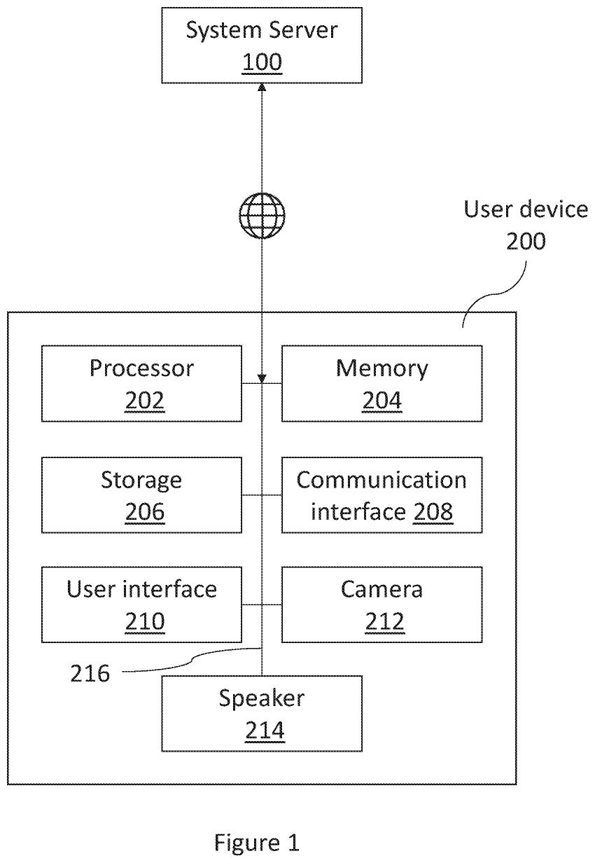

FIG. 1 shows an exemplary system overview for implementing the present invention. The computer instructions for implementing the measuring tool instructions are stored in a system server 100. The server 100 sends these instructions over the internet in order to facilitate the running of the measuring tool via a web browser on a remote user device 200. A user at any location can access the tool via the exemplary user device 200 at a remote location. The user device 200 is a computer comprising a processor 202, a memory 204, a storage 206, a communication interface 208, a user interface 210, a camera 212, and (preferably) a speaker 214. These components are coupled to one another by a bus 216. The user device 200 may be a desktop computer, a laptop computer, a mobile phone, tablet or any other computing device. The processor 202 is a computer processor, such as a central processing unit (CPU) or a graphical processing unit (GPU). The processor is arranged to execute instructions in the form of computer executable code, including instructions stored in the memory 204 and the storage 206. The instructions executed by the processor include instructions for coordinating operation of the other components of the computer device 200, such as instructions for controlling the communication interface 208, the camera 212 and the user interface 210. In various embodiments, the processor comprises one or more of a field programmable gate array (FPGA), a coarse grain reconfigurable array (CGRA), or an application specific integrated circuit (ASIC). It will be appreciated that a variety of other circuits may be used for the processor to perform operations and/or implement methods using software or hardware.

The memory 204 comprises random access memory (RAM) that is available for use by the components of the computer device 200. The memory may be a volatile memory, for example in the form of an on-chip RAM integrated with the processor 202. Typically, the memory is separate from the processor. The memory is arranged to store the instructions processed by the processor in the form of computer executable code. Typically, only selected elements of the computer executable code are stored by the memory at any one time (e.g. the computer executable code may be stored transiently in the memory whilst the processor is executing a process).

Implementations of aspects of the measuring tool of the present invention comprise running some processes locally on the processor 202 according to instructions sent via the communication interface 208 and stored in the memory 204. Typically, initial technical checks of the user device 200 are performed to ensure that the technical requirements are met in order to run the measuring tool.

The storage 206 comprises non-volatile memory. The storage may be embedded on the same chip as the processor 202 and/or the memory 204. Equally, the storage may comprise an embedded or external form of memory.

The communication interface 208 enables the computer device 200 to communicate with other devices. The communication interface may comprise one or more of: a wired communication interface, such as an Ethernet interface; a short-range wireless communication, such as a Bluetooth® interface or a Wi-Fi interface; and a long-range wireless interface, such as a 2G, 3G, 4G, or 5G interface.

The user interface 210 enables the user to interact with the user device 200. The user interface typically comprises one or more of: an input/output device; a display; a speaker; a mouse; a keyboard; and/or an interactive touchscreen.

The user device 200 of the present invention comprises a camera 212 to enable photos and/or video to be recorded, from which measurements can be determined. Additionally, the user device preferably further comprises a speaker 214, which allows sound to be output. Audio instructions, for example how to position their head for correct measurements, can be output to the user via the speaker 214. The camera 212 and/or speaker 214 could be provided external to the device 200 but provided in connection to it, in order to achieve the same purpose. For example, an external webcam may be connected to the device 200 to act as the camera 212. In the present invention, the user device 200 accesses the measuring tool via the communication interface 208, and displays it to the user via the user interface 210. The measuring tool requires photographs of the user's face in order to obtain the required measurements, and so comprises instructions to facilitate access to the camera 212 of the user device 200. Typically a request to access the camera will be output to the user, to which they must agree, before access can be granted. The camera feed from the camera 212 is displayed on the user interface 210 via the web browser window, so that the user can view it and adjust their positioning. Verbal audio instructions may be output to the user via the speaker 214 and/or verbal and/or graphic visual instructions may be output to the user via the user interface 210.

### Browser Software Structure

Providing access to the measuring tool via a web browser improves the ease of access for a user. For example, a user is unlikely to order bespoke loupes very frequently, and so it would be cumbersome and inefficient to need to download a tailored application or install specific software for running the measuring tool. However, in order to achieve the necessary measurement precision and accuracy to ensure small loupes with high magnification telescopes can be fitted precisely, the present invention utilizes orientation processing to ensure the correct position of a user's head from which measurements can be extracted, in addition to calibration processes (as will be described in detail later). In order to ensure sufficient processing power is devoted to all the processes of the measuring tool, some of the software processes are run locally at the user device 200, while others are performed centrally on the server 100.

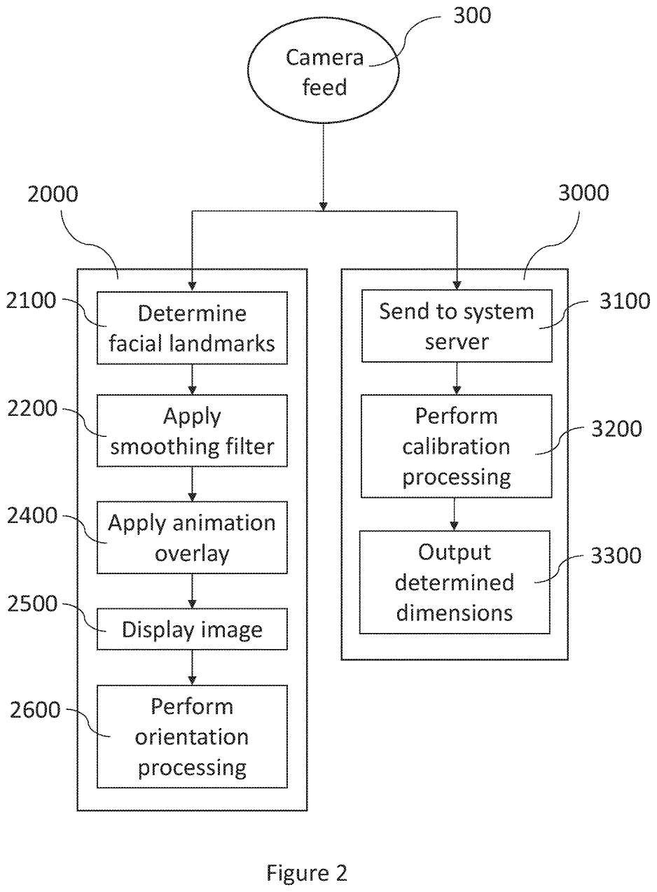

An exemplary software structure is shown in FIG. 2. A camera feed 300 from the camera 212 of the user device 200 is duplicated, and a first copy processed locally at the user device 200 and a second copy processed centrally at the system server 100.

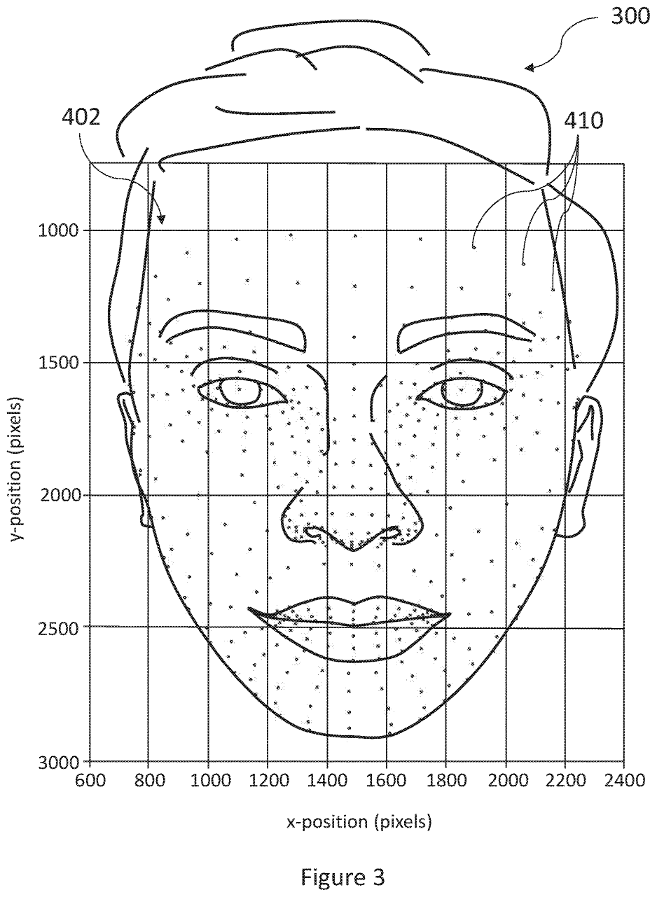

A first process flow 2000 is performed locally on the first copy of the camera feed 300 by the processor 202 of the user device 200, according to instructions sent via communication interface 208 to the memory 204. As a first step, landmarks on a user's face are determined 2100 in real time. Then, a smoothing filter is applied 2200 to smooth the positions of the landmarks over time to prevent unwanted ‘jitter’ in the display. The landmarks are then used to create an animation of the user's face 2400, taking the form of a face mesh. The landmarks and the animation are displayed on the camera feed 300 of the user's face, overlaid in real time when displayed 2500 on the user interface 210 via the browser window. FIG. 3 shows an example of the display 400 showing the user's head 402 with the landmark points 410 and the face mesh 420 provided as an overlay. Finally, orientation processing is performed 2600 to ensure the user's head is in the optimum position, including outputting prompts to a user to correct the positioning. (These steps will all be described in detail later). An image is recorded automatically when the user is in the correct position. The correlating frame of the raw camera feed 300, which is sent to the system server 100, is then used to determine the required dimensions via a calibration process.

A second process flow 3000 is performed on the second copy of the raw camera feed 300 sent to the system server 100. Firstly, the second copy is sent to the system server, 3100, and then calibration processing 3200 is performed. The calibration processing 3200, as will be described in detail later, calibrates the pixel dimensions of the camera feed 300 to real dimensions of the user's face in order to output the physical measurement dimensions required to manufacture the bespoke loupes 3300. The second process flow 3000 is performed on a second copy of the camera feed 300 to ensure that the calibration processing 3200 is applied to a high resolution, raw version of the images.

### Local Processes Overview

A first process flow 2000 is performed locally at the user device 200 on the first copy of the camera feed 300. As an initial step 2100, the user device 200 implements an algorithm on the camera feed 300 to locate facial landmarks. For example, the Mediapipe machine learning library can be used to determine 468 facial landmarks with high accuracy in real time. This employs machine learning to infer the 3D facial surface. Facial landmarks are features such as eyes, mouth corners, lips, ears etc., which may be used as landmarks to determine the position of a face within an image. Facial landmarks may be determined by first processing the frame using a lightweight face detector to produce face bounding rectangles and identify several initial landmarks (e.g. eye centers). The lightweight face detector may be a machine learning model, such as BlazeFace. The face bounding rectangles are used in conjunction with the initial landmarks to crop the face from the original image, and to resize it for input into a mesh prediction neural network. The mesh prediction neural network may be trained on a large number of facial images. The mesh prediction neural network produces a map of landmark coordinates, which are then mapped back to the original image coordinate system. FIG. 3 shows a representative camera feed 300 containing an image of a user's face 402. Facial landmark points 410 which have been determined are shown overlaid on the image.

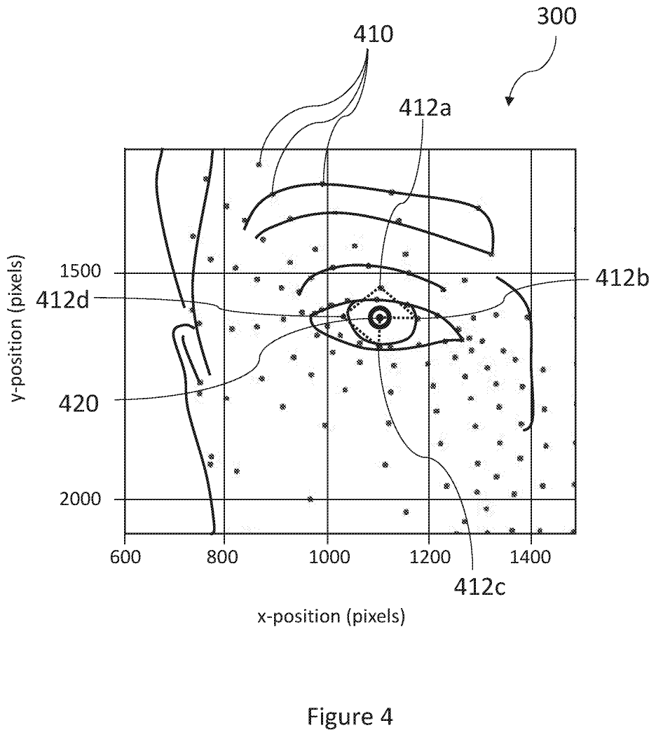

In order to determine the centre of each of the pupils 420 of the eyes in the user's face 402, the system performs an interpolation of surrounding landmark points 412a, 412b, 412c, 412d, as shown in FIG. 4. In particular, these landmark points 412a, 412b, 412c, 412d form a diamond around the pupil, and typically are the four nearest landmark points. The centre of the diamond formed by the landmark points 412a, 412b, 412c, 412d is determined to locate the centre of the pupil. FIG. 4 shows a magnified portion of the camera feed 300, over which is laid the landmark points 410 and which shows the interpolation of landmark points 412a, 412b, 412c, 412d around the eye, used to determine the centre of the pupil 420.

As the determination of landmark points occurs in real time, there can be observed some jitter in the locations of the landmark points 410. This can typically be due to slight movements of a user between frames of the camera feed. At step 2200, the coordinates of the landmark points 410 in the original image coordinate system are smoothed using a smoothing filter which averages position over time. In particular, a normalized position of each landmark point 410 over time can be determined. By way of example, this may be implemented using a One Euro filter.

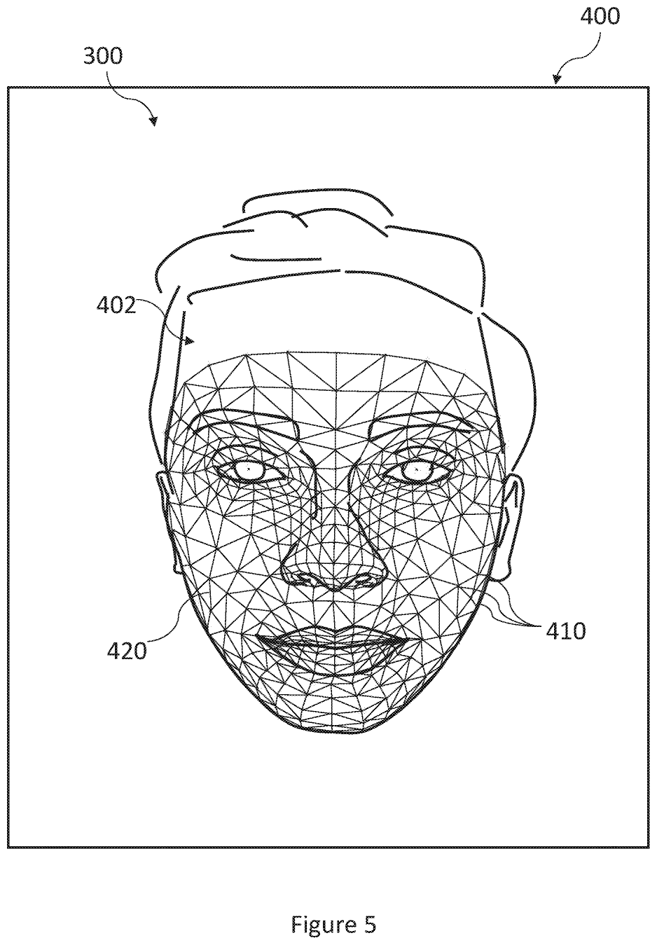

At step 2400, an animation overlay is provided over the image feed. As shown in FIG. 5, this typically comprises a face mesh 420, which is formed using the smoothed positions of the landmark points 410. This effectively provides a topology visualization for the user's face.

At step 2500, the camera feed 300 is displayed to the user via the web browser shown on the user interface 210 of the user device 200. As shown in FIG. 5, the camera feed 300 is displayed with an overlay comprising the smoothed landmark points 410 and the face mesh 410 animation. The animated overlay is adjusted for each frame in the feed, so that the animation overlay continues to cover the user's 402 face even as the user moves within the frame i.e. the landmark points 410 and face mesh 420 follow the movement of the user's face 402. The web browser window displayed on the user interface 210 typically further comprises a dot (or other similar marker), on which the user can focus, to help to ensure that they continue to look straight ahead (in particular, the pupils of their eyes continue to look straight ahead).

At step 2600, the system performs orientation processing live by implementing an orientation algorithm. In this step, a user's facial orientation is determined by reference to the facial landmark points 410. This is used to ensure that the user's face 402 is in a correct position (for example, looking straight ahead at the camera 212) so that accurate measurements can be determined.

### Orientation Processing

In order to determine accurate measurements of the user's face, the measurement tool needs to acquire good quality images of the user's face 402. In this context, good quality images have a good resolution and show the user's face 402 clearly looking straight at the camera, occupying a sufficiently large area of the image to show sufficient detail but ensuring that all features are included (and no distortions occur due to too close proximity to the camera). In order to obtain such a good quality image, orientation processing is performed to check whether a series of defined parameters are within defined acceptable ranges. This is typically performed in real time, and provides feedback to the user. If all the parameters are within an acceptable range, an image is captured. If one or more of the parameters falls outside an acceptable range, instructions are output to a user indicating how to rectify their position. These instructions can be audible and/or visual. For example, the user may hear a voice instructing them how to rectify their orientation. Alternatively or additionally, a user may see written instructions and/or graphic instructions (such as arrows) on the display.

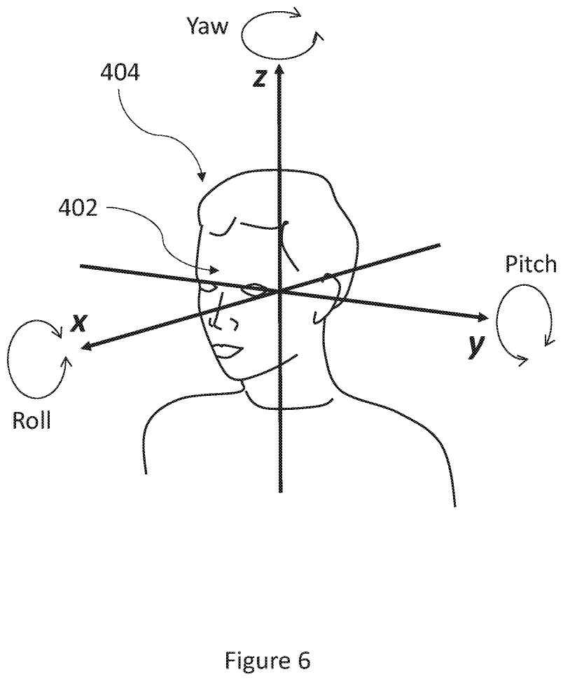

The orientation of a user's face may vary in three dimensions: roll, pitch, and yaw. These orientations are illustrated with reference to a user's head 404 and face 402 in FIG. 6. In this frame of reference, the x-axis corresponds to the direction parallel to the direction in which a user's face 402 looks straight ahead. The y-axis is parallel to the line joining the ears on the user's head 404, and the z-axis is parallel to the line extending directly upwards or downwards from the top of the user's head 404. As such, when imaging a front view of the user's face 402 (i.e. the user looking straight ahead into the camera), the camera 412 should be located along the x-axis relative to the user's face 402, and the image will show the y-z plane. ‘Roll’ refers to rotation around the x-axis; ‘pitch’ to rotation around the y-axis; and ‘yaw’ to rotation around the z-axis. Accordingly, ‘roll’ of a user's head 404 refers to tilting relative to a plane disposed symmetrically between the right and left shoulder (such that a large roll brings the user's ear towards the adjacent shoulder). When the camera 212 is positioned along the x-axis to take a front-view image of the user's face 402, the roll is seen as rotation of the user's head within the plane of the camera image. The pitch of a user's face 402 refers to the extent to which it is pointing upwards (lifting the chin upwards) or downwards (lifting the chin downwards, for example to look at the user's feet). The yaw of the user's head 404 refers to the extent to which it is turned to the left or to the right, for example (when moved to a large extent) to look over the user's left shoulder or right shoulder.

Since the camera feed 300 is generated using a single camera 212, no depth measurements are typically taken. This is not a problem if the measurements of the distance between the eyes are only taken within the x-y plane, i.e. if the face is oriented such that the plane of the user's face 402 is parallel to the plane of the camera (the user is looking directly into the camera 212). It is therefore important to determine the orientation of the user's face 402, and to instruct the user to adjust their facial orientation if necessary in order to obtain suitable images from which measurements can be determined.

The system analyses landmark features of the camera feed 300 of the user's face 402, using the landmark points 410, to determine the orientation of the user's face. In particular, analysis is used to determine the extent of any roll, pitch or yaw of the user's face. This includes the calculation of parameters including:
Interpupillary distance (IPD)
Monocular pupillary distances (PDs)
Face width
Face height
Top to bottom angle
Left to right angle
Left cheek to nose angle
Right cheek to nose angle.
Area of the left silhouette
Area of the right silhouette
Ratio of area of left silhouette to right silhouette

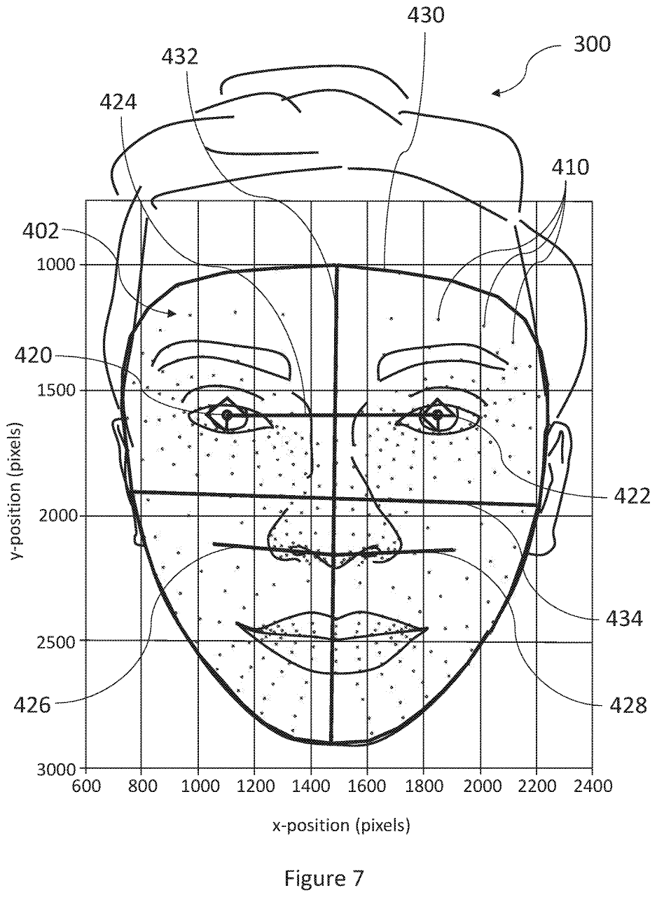

FIG. 7 shows a camera feed 300 of a user's face 402, over which is displayed the landmark points 410. The landmarks points are analysed to determine pixel distances for some of the parameters listed above. In a simple manner, the IPD 424 is defined by a line between a first pupil centre 420 and a second pupil centre 422 (the pupil centres having been defined by the interpolation method as previously described). More precise values for distances between each pupil centre and a defined fixed datum, such as the bridge of the nose, are preferably also determined, to achieve a precise fit for the loupes. Such a distance is known as a monocular pupillary distance, PD, and as there is typically asymmetry in the pupil centre positions, determining the monocular PDs can ensure accurate location of the telescopes on the lenses. The fixed datum may typically be defined using a reference, such as a marker on reference frames, as will be described in a later section. Alternatively, a central point of the nose bridge of a user may be defined, and the monocular PD determined with reference to it. The perimeter 430 is defined, and a line from the top point to the bottom point defines the length 432, while a line from left to right defines the width 434. A left nose to cheek line 426 is provided between a defined left cheek point and a nose point, and similarly, a right nose to cheek line 434 is provided between a defined left cheek point and a nose point. (‘Left’ and ‘right’ in this sense are defined as is viewed on the screen, and, as the user is viewing a mirror image on the screen, this correlates to the user's ‘left’ and ‘right’.)

Acceptable ranges for defined parameters have been determined by calculating value parameters for a series of photographs used as training data. (In an exemplary implementation, these are photographs of users which have been taken by specialists when fitting loupes manually.) Acceptable ranges include tolerance values, as user's faces typically have some normal asymmetry. In one embodiment, the tolerance values are determined by classifying training images as “suitable” or “unsuitable” for processing, and then rejecting the 5% most extreme values of the “suitable” images. The remaining value range is then determined to define acceptable values. For example, the angle of a line between the eyes of a user may be found to be at an angle of 2 degrees to the horizontal. Analysis of the training data may show that 95% of the “suitable” images have an angle between-3.22 degrees and 2.59 degrees. Since 2 degrees falls within this range, it may be deemed an acceptable value. In alternative implementations, the acceptable ranges may be defined manually. If all the determined parameter values are within their respective acceptable ranges, the system goes ahead and records an image. If any of the parameters are determined to be outside of their acceptable range, audio and/or visual instructions are output to a user relating to how they should amend the orientation of their head 404.

As a first check, it can be beneficial to determine whether the user's head 404 is at an acceptable distance from the camera 212. The interpupillary distance (IPD) (and the monocular PDs) can vary dependent on where a person is looking: if a person is looking into the distance, then the IPD typically expands, while if a person is looking at an object close-up (for example, reading a book), then the IPD typically contracts. It has therefore been found by the inventors that it is beneficial to ensure that users maintain a consistent distance from the camera, i.e. a consistent focal point and focal distance of their gaze. Keeping this consistent can assist in facilitating accurate determination of the IPD and monocular PDs for each user. The IPD and monocular PDs are naturally different for different people, and by keeping the focal distance of the user's gaze consistent, the variations in determined IPDs and monocular PDs can be considered to be due to individual anatomical variations, and any effects of different focal distances can be at least minimized (preferably eliminated).

It has been found that a distance of 400 mm between the camera and the user's eyes is preferably used. This is because it corresponds to an average arm length of different users, fitting well within the anthropometric profile of most people worldwide. The relationship of the distance from the object to the camera, D, the focal length of the camera lens, f, the actual width of the object, W, and the width of the object in the image captured by the camera (as determined by converting the pixel dimensions to physical length), w, is:

$$D = \frac{f \cdot W}{w}$$

The focal length, typically measured in millimeters, describes how a lens captures an image, affecting the angle of view and magnification. However, the focal lengths typically vary between different devices, which can introduce challenges in distance estimation. A range for typical focal lengths of different smartphones has been found to be 23-26 mm (with some as high as 32 mm). This provides a baseline for distance estimation.

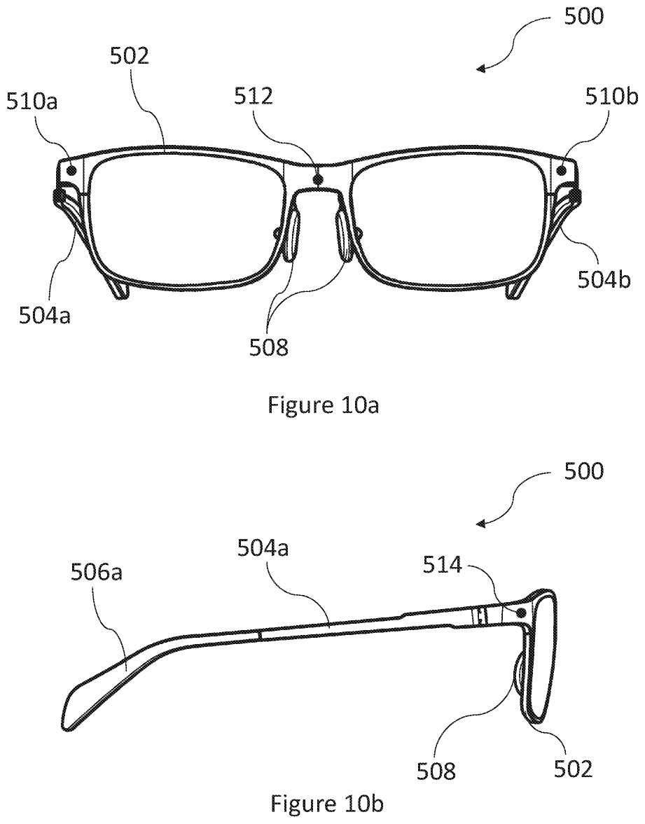

Different methods may be employed for determining the object width. One approach can be to identify an object which has a known dimension (for example, known width). Such an object may be a calibration marker; for example, specific markers on calibration frames (which are illustrated in FIGS. 10a and 10b and will be described in further detail in a later section). The reference markers are a known distance apart (for example, 100 mm) and this known distance acts as the reference object width for the purpose of determining the distance between the object and the camera. This typically requires real-time processing. As an alternative object dimension, the diameter across a user's iris can be used. As there is a consistent value of iris diameter across the population (11.71 mm), this presents a reliable dimension. Additionally, this can be used to determine the distance to the user's eye, which can aid in achieving an accurate measure for the interpupillary distance (IPD). It can be preferable to use high-resolution images for determinations using the iris diameter, as this is a small dimension relative to a user's facial features. A high-resolution image is typically defined as having a resolution of at least 300 pixels per inch (PPI) and/or 1 MPx, while preferably a high-resolution image used in the described method has a resolution of at least 5 MPx, more preferably at least 7 MPx.

A further method comprises determining a ratio between the area of the user's face 402 and the size of the total image 400. This can be calculated by calculating the total area within the perimeter 432, and the total area of the image 400, and then determining the ratio. This can enable a distance estimation to be performed based on how much of the frame the face occupies. If a user is positioned too far away from the camera, their face 402 is displayed smaller relative to the total area of the display 400. This may result in the image 400 containing insufficient information on the user's face 402 for accurate determination of facial measurements. However, when the user is too close to the camera, this may result in their face 402 not fitting within the frame, meaning some features are not available for analysis. The system may therefore determine the area of the user's face 402 relative to the total area of the display image 400. This method adapts to variations in facial coverage relative to camera distance, offering a practical approach for distance estimation. In an exemplary embodiment, the acceptable range is defined as a ratio of between 0.0821 and 0.4367. If the ratio for an image is found to be below the lower threshold, the user may be prompted to move towards the camera (for example, a prompt is output to the user interface 210 and/or speaker 214). If the ratio for an image is found to be above 0.4367, the user may be prompted to move away from the camera. These (and all) prompts may be audible and/or visual. Of course, movement of the user relative to the camera is sufficient-so the user and/or the camera may physically be moved. The prompts typically guide a user to achieve the desired 400 mm distance (although of course other distances may also be chosen).

Additionally or alternatively, the system may determine a height to width face ratio for the image. This can be determined by calculating the ratio of the height line 432 to the width line 434. Some variation of the height to width ratio of faces occurs naturally between people. However, extreme height to width ratio values may be indicative of a problem within the image. For example, a user wearing a face mask which occludes the lower portion of their face may receive an anomalously low height to width value, due to the facial processing algorithms concluding that the mask marks the bottom of the face. Since the facial processing algorithms are trained only on faces that are not obscured, the loss of information regarding the nose and mouth can result in problems, such as the algorithm positioning these landmarks incorrectly on exposed portions of the face, or incorrectly extrapolating into the mask area. Errors in the assignment of nose and mouth position may affect the assignment of other landmarks within the face. It is therefore desirable to instruct a user to uncover their face if a mask is found to be obscuring a large portion of their face. In some embodiments, the system outputs audible and/or visual prompts, for example via the user interface 210 and/or speaker 214, to instruct the user to remove a face mask or equivalent face covering.

An anomalously high height to width ratio may also be indicative of a problem with the image. It is known that objects that are closer appear larger than objects that are further away. When an image is taken at a suitable distance from the user, the difference between the foremost points in a user's face and the hindmost points in a user's face is not sufficiently significant to result in any visible distortion of the face. However, when the camera is too close to the user's face, the image may show visual distortion, rendering landmarks such as the nose much larger. This distortion will also increase the height to width ratio of the face, because the forehead, being closer to the camera, will appear much larger, and the side edges of the face, being further backward, will be reduced in size. Since it is desirable to minimize such distortion to facilitate accurate measurements to be taken from the image, the system may prompt a user to move further away from the camera when an anomalously high height to width ratio is determined.

In some instances, the system may evaluate the image resolution. When the image resolution is below a threshold value, it is not possible to accurately determine the position of facial landmarks. Accordingly, the system evaluates the image resolution and, if the image resolution is below a threshold, the system may request that the user use a different camera for the process. In one example, a minimum threshold value is 7 MPx. The system may also in some embodiments prevent the user from zooming in too much. The use of video imaging rather than static imaging can advantageously provide a consistent frame size (in pixels). In some embodiments, metadata is extracted to check the focal length and/or zoom of the camera.

The system may also evaluate a face height (length of height line 432) relative to an image height. Additionally or alternatively, the system may evaluate a face width (length of width line 434) relative to an image width. Acceptable tolerances for these may be defined manually or may be defined via analysis of training data sets.

Once the user is at the correct distance from the camera, they also need to orient their head so that they are looking straight ahead at the camera (i.e. roll, pitch and yaw are minimized to the greatest extent possible). This is to ensure any measurements are accurate.

The extent of roll of the user's face 402 can be correlated to the angle between a line from left to right (the width line) 434 relative to the horizontal, and/or the angle between a line drawn from the top centre to the bottom centre of a user's face 404 (the length line 432) relative to the horizontal. By way of example, the tolerance ranges defined using the training data may be between −3.22 degrees and 2.59 degrees for the first value, and between 86.6 degrees and 92.6 degrees for the second. If the values calculated during the orientation process are within the acceptable limits, then no correction is required (and if all other parameters are also within limits, the system can progress to capturing the image). When the system determines that a feature is outside acceptable limits, the system may prompt a user to reorientate their head. For example, if the width line 434 is found to be 8 degrees relative to the horizontal, the system may prompt the user to reposition their head by tilting it to the appropriate side. The prompt may be visual and/or audible. For example, a user may hear and/or see displayed on the screen “tilt your head to the right”. Alternatively or additionally, arrows indicating the direction in which the user must tilt their head may be output to the display.

The pitch of the user's face 402 can be correlated to nose to cheek angles. The left nose to cheek angle is the angle between the left nose to cheek line 426 and the horizontal, and the right nose to cheek angle is the angle between the right nose to cheek line 428 and the horizontal. This angle may then be compared to tolerance values determined from training data. In one embodiment, the training data may have 95% of images having a nose to right cheek angle of between-17.05 degrees and 2.81 degrees, and so angles within this interval may be deemed acceptable. In one embodiment, both the right cheek and left cheek angle values must fall within the acceptable respective ranges. Alternatively and/or additionally, an average angle for both cheeks must fall within the acceptable range for average values. When the angle is found to be outside the acceptable range, the system may prompt a user to tilt their head forwards or backwards as appropriate. This prompt may be audible and/or visible.

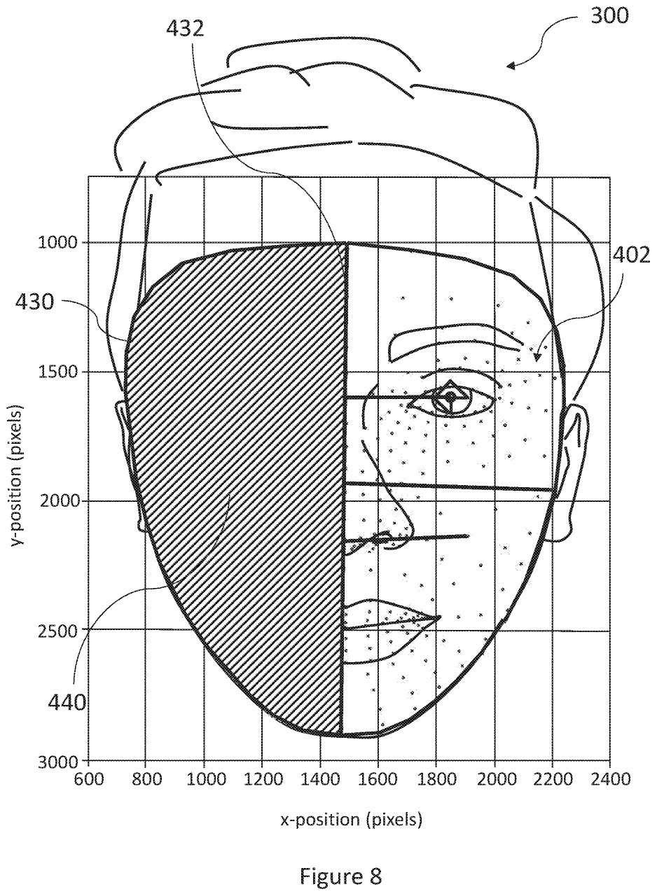

The yaw of a face may be determined by comparing the area of the right side of the face (the ‘area of the right silhouette’) to the area of the left side of the face (the ‘area of the left silhouette’). When a user is facing the camera straight on with no yaw, the area of the right side and the area of the left side should be approximately equal. When a user has angled their head towards one side (for instance facing slightly to the right), the areas will not be equal. In one embodiment, a representative value for the yaw is calculated according to the following formula:

$$ϕ = ( 1 - \frac{{A}_{right}}{{A}_{left}} ) * 100$$

Where φ is the representative value for the yaw, Δ_{left} is the area of the left side, and Aright is the area of the right side. FIG. 8 shows how the area of the left side can be calculated—it is defined as the area located to the left of the height line 432 within the perimeter 430 (shown shaded in FIG. 8). The area of the right side can be calculated in a corresponding manner as the area to the right of the height line 432 within the perimeter 430. In an exemplary embodiment, the allowable values for the representative value for the yaw may be defined as between −15.51 and 11.09. When the representative value for the yaw falls within this range, it can be deemed allowable. When the representative value for the yaw falls outside this range, the user is instructed to angle their head to the left or right as appropriate via audio and/or visual outputs.

When the user has oriented their head 404 and face 402 such that all the above measurements fall within defined acceptable ranges, the system then captures an image. The corresponding raw video feed of this frame is processed at the system server 100. This includes calibration processing 3200, in order to determine physical facial distance measurements from the pixel measurement values. In some preferable implementations, when the system has determined that all the relevant measurements fall within defined acceptable ranges, the system captures a high-resolution still image via the camera 212, before then proceeding to video. The high resolution still image can effectively form the primary frame of the video. This is sent to the system server 100 for processing. This can ensure that the primary frame is always high resolution, which can be beneficial for processing to determine facial distances (such as interpupillary distance, IPD, and relative pupillary height, PH). A high-resolution image is typically defined as having a resolution of at least 300 pixels per inch (PPI) and/or 1 MPx, while preferably a high-resolution image used in the described method has a resolution of at least 5 MPx, more preferably at least 7 MPx.

Some or all of the criteria parameters may be used in any combination and in any order. Additional parameters and associated criteria may also be employed.

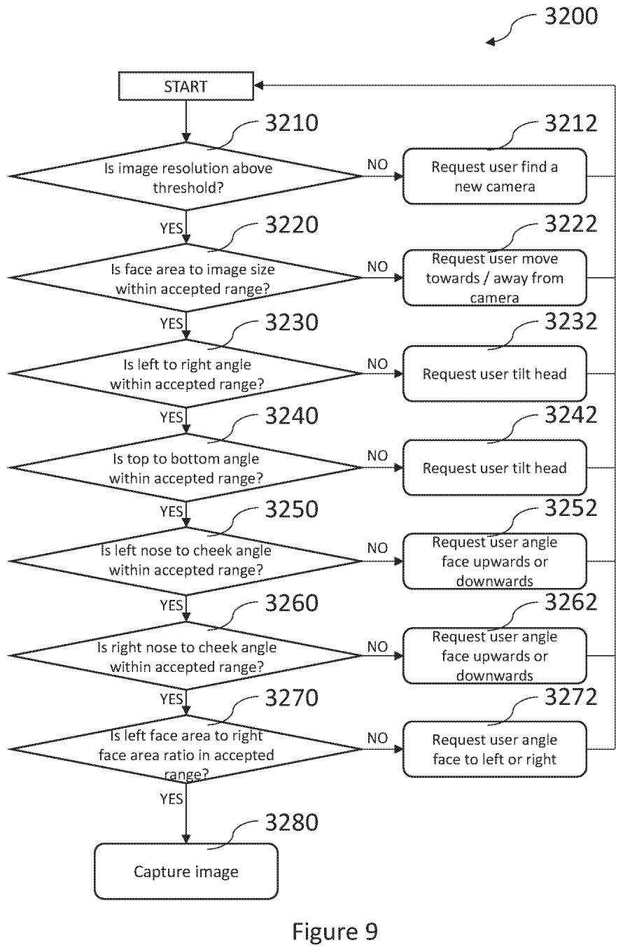

FIG. 9 shows a workflow of an exemplary orientation process workflow 3200. The system determines whether each parameter is within an acceptable value range: if it is, it moves on to the next step; if it is determined not to be within the acceptable range, then instructions are output to a user indicating how to correct the issue. In this exemplary embodiment, a first step comprises determining whether the image resolution is above a threshold value 3210, and if it is not, a request that the user uses a new camera 3212 is output. If the resolution is acceptable, the system moves to the next step of determining whether the ratio of the area occupied by the user's face to total image area is within an acceptable range 3220. If it is not, a request is output that the user move towards or away from the camera 3222 (depending on whether the ratio is below or above the accepted range). The system then reverts to the start of the workflow. If the ratio is within the acceptable range, the system moves to the next step of determining whether the left to right angle (the angle of the width line 434 to the horizontal) is within the accepted range 3230. If it is not, this indicates roll of the head, and so a request is output that the user tilts their head 3232. The system then reverts to the start of the workflow. If the left to right angle is within the accepted range, the system moves to the next step of determining whether the top to bottom angle (the angle between the length line 432 and the horizontal) is within the acceptable range 3240. If it is outside of the acceptable range, this can also indicate roll of the head, so a request that the user tilts their head 3242 is output, and the system reverts to the start of the workflow. If the value is deemed within the acceptable range, the system moves to the next step of determining whether the left nose to cheek angle is within the defined acceptable range 3250. The nose to cheek angles are indicative of whether there is pitch of the head. If the value is outside of the acceptable range, a request that the user tilts their head upwards or downwards 3252 is output (depending on whether it is above or below the acceptable range), and the system reverts to the start of the workflow. If is it determined to be within the acceptable range, the system moves to the next step of determining whether the right nose to cheek angle is within the defined acceptable range 3260. If it is not, a request that the user tilts their head upwards or downwards 3262 is output (depending on whether it is above or below the acceptable range), and the system reverts to the start of the workflow. If is it determined to be within the acceptable range, the system moves to the next step of determining whether the ratio of left area of the face to right area of the face is within the acceptable range 3270. If it is not, this indicated roll of the head, and so a request that the user move their head to the left or right 3272 (in dependence on which side has the greater area) is output. This flow occurs in real time, outputting instructions on how to adjust the user's orientation until all of the parameters are within acceptable threshold values, at which point the image frame is ‘captured’ 3280 for calibration processing.

In some instances and implementations, it can be beneficial to obtain further images of different angles of the user's face. These can be used to develop a 3D model/avatar of the head and eyes. Using a 3-dimensional image rather than 2-dimensional image can help to prevent any errors that may be introduced when going from a 3D object (i.e. the human head and eyes) to a 2D object (i.e. an image on a screen). This can assist in calibration and in determining measurements of the face, such as vertex, both the methods of calibration and measurement will be described in further detail in later sections. A series of images of the user's face from different angles is obtained by moving the camera and the face relative to one another. This can be achieved either by the user rotating their face, or by the camera moving around the user (for example, a user may keep their head still but move a mobile phone around their head, the mobile phone recording images). The images may be captured as video images, as a series of static photographs, or a combination of both. In some examples, a series of eight images is captured, each showing the user's face from a different angle.

### Calibration Process

For the manufacture of bespoke magnification loupes, physical dimensions of a user's face must be determined. The required dimensions include interpupillary distance (IPD), which is the lateral distance between the centre of the pupils of each of the user's eyes; relative pupillary height (PH), which is the relative height of the user's two pupil centres (it is normal for eyes to not be completely symmetrical and be positioned at slightly different heights); and vertex, which corresponds to the distance between the aperture of the telescopes and the wearer's eyelid. Preferably, monocular PDs are also determined—this is the distance from a fixed data point (typically central to a user's face) to each of the pupil centres, and so there is a value for the left eye and a value for the right eye. Typically, images with a front view are used to perform IPD and relative PH measurements, and images with a side (profile) view are used for vertex measurements. In order to determine the physical distances for these measurements, the pixel dimensions in the images must be calibrated.

As described previously and as shown in FIG. 2, a raw copy of the camera feed 300 is sent to the system server 100 for calibration processing 3200 via a calibration algorithm. This reduces the processing capacity required of the user device 200. A high-resolution raw copy of the correctly oriented frame of the camera feed 300 is used for calibration in order to achieve good precision and accuracy of the determined dimensions output.

In order to calibrate the images from pixels to physical dimensions, it is necessary to correlate the dimensions in the image to the pixels forming the image. This can be achieved in a number of different ways, as will be described.

### Using a Credit Card

In a first example, a reference object of known physical dimensions is used to correlate the pixel dimensions to physical dimensions. Credit cards, and in particular the strip on one side, are of a standard size and an object most users are likely to have to hand. A user typically holds a credit card up to their forehead while the camera is recording images. The pixels dimensions of the credit card and its strip can be used to determine the correlation between pixels dimensions and physical dimensions, thereby calibrating the images. The dimensions of the landmarks on the face, such as interpupillary distance (IPD), can then be determined by multiplying the pixel dimensions with a determined correlation factor.

In some implementations, in order to further minimize error, the system may be further configured to detect credit cards in multiple planes and orientations. As the dimensions of the credit card are known, the pixel dimensions of the credit card within the image can be correlated to these known physical dimensions to determine orientation. For example, when the card is positioned at an angle, the relative dimensions in this perspective view, as will be seen in image and translated to pixel dimensions, can also be known (for example, angles of the side edges, height to width ratio etc.). This can be used to calibrate and in determining orientation. The strip on the credit card can be used as an anchor point to assist in determining orientation.

### Using References Frames

In a second example, a user may be sent a pair of ‘reference frames’ to wear when using the measuring tool, which acts as the reference object of known physical dimensions. The reference frames may typically be used when higher accuracy is required and allowable tolerances are smaller, for example for high magnification loupes. Example reference frames 500 are shown in FIGS. 10a and 10b, from a front view and a side view respectively. These resemble typical glasses (or indeed the frames which hold telescopes to form loupes) and comprise a frame 502 which includes a bridge and carries two nose pads 508 for resting on the nose. The end pieces of the frame 502 are connected via hinges to two arms 504a, 504b, including temple tips 560a, for resting on a wearer's ears. The user can adjust the nose pads 508 to make the frames 500 comfortable for their own use. The position the user wears the reference frames 500 can therefore mimic the position in which they will wear the loupes, which can aid in determining relevant dimensions for the manufacture of those loupes. The frame 502 may carry clear lenses or may be provided empty.

The reference frames 500 comprise markers of known location which can be used in calibration. In the implementation illustrated in FIGS. 10a and 10b, the markers are provided as dots on the frame 502. These include markers 510a, 510b located on end pieces and a marker 512 located on the nose bridge, all of which are in view when viewing the reference frames 500 from the front (as is shown in FIG. 10a). Markers 514 are also provided on the arms 504a, 504b, which are in view when viewing the reference frames 500 from the side, as is shown in FIG. 10b. The markers 510a, 510b, 512, 514 can therefore provide calibration markers for images taken from a front view and images taken from a side (profile) view. Typically, the front view is used to determine IPD and PH, and the side view is used to determine vertex.

In other implementations, the reference frames 500 alternatively or additionally comprise laser-etched lines spaced at defined distances from one another (for example, 1 mm). These markings can provide a physical measurement scale which can be used for calibration.

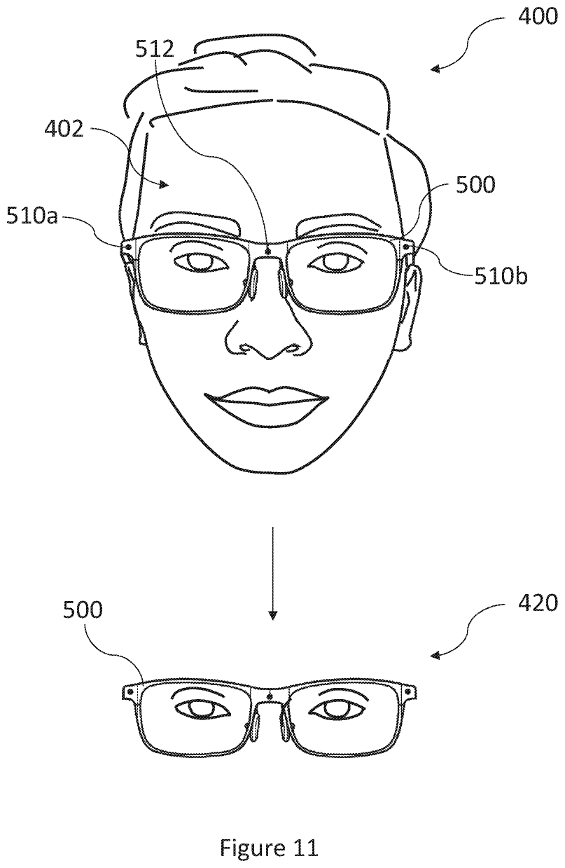

In some implementations, the calibration process first comprises the step of extracting the portion of the image containing the reference frames 500. This can be implemented autonomously using an image segmentation model (for example the ‘Segment Anything’ model by Meta®). FIG. 11 shows the display 400 including a user's face 402 wearing the reference frames 500. FIG. 11 also shows that a segment 450 of the display, comprising only the reference frames 500, is extracted. The dimensions of the reference frames 500 are known, and so the segment 450 can be used to correlate the pixel dimensions of the image with physical dimensions. This provides a much cleaner image on which to perform the calibration processing.

Additionally, the pixel dimensions can be used to determine and/or verify orientations of the user's head within the image. The exact dimensions of the reference frames 500 are known, and the reference frames 500 are known to be perfectly symmetrical, while human faces typically have asymmetries which can reduce the accuracy with which determinations of the orientations can be made. For example, a human face may have one cheek which is slightly larger than the other—in which case, the user may be looking straight ahead at the camera but the ratio of the left area and right area might indicate head yaw. By contrast, the reference frames 500 are known to be symmetrical. Therefore, if the segment of the reference frames 500 is not symmetrical, it is more likely that the user is not orientated in the optimum position for measurement. For example, if the distance between a first side marker 510a and the bridge marker 512 is greater than that between the second side marker 510b and the bridge marker, it is indicative the first side is closer to the camera and so there is very likely to be head yaw. As another example, human faces have a wide range of aspect ratios while the aspect ratio of the reference frames 500 is precisely known. Therefore, if the aspect ratio of the segment image of the reference frames 500 does not correlate to the known value, it is an indication that the head may not be at the optimum pitch.

Additionally, the central marker 512 on the nose bridge can be used as the fixed datum from which to measure the monocular PDs. In such an instance, a first relevant value is the distance from the central marker 512 to the left eye pupil centre and a second relevant value is the distance from the central marker 512 to the right eye pupil centre. The direct distance from the marker 512 to each eye centre and/or the horizontal and vertical components of the vector between the marker 512 and each eye centre can also be determined. For loupes, it is important that the telescopes are placed in the correct positions for the user's pupils, and so it can be important to determine the monocular pupillary distances from correctly oriented images. It can also be useful to determine this relative to a point on reference frames being worn by the user, as this can give an indication of how the loupes will sit on the user's face.

### Using LIDAR Technology

In a further implementation, Light Detection and Ranging, or Laser Imaging, Detection and Ranging (LIDAR) technology can be used for calibration. Some newer models of phones and tablets are loaded with a LIDAR scanner which can be used in this implementation. Alternatively, LIDAR scanners could be provided.

A LIDAR scanner emits a light from a laser, which is reflected from the relevant surface back to the scanner, where a sensor detects it. This capability can be used to determine the distance at which objects are located. This distance can then be used to calibrate the pixel dimensions in order to obtain physical measurements without the need for a reference object of known physical dimensions.

In some implementations, this technology can also be used to overlay virtual measurement frames over the bridge of the nose in the image accurately. In some implementations, this technology can potentially also be used to show the user the loupes for which they are being fitted on their face, in effect creating a virtual ‘try-on’.

### Depth Analysis

In some implementations, it can be beneficial to determine the depth or distance from the camera, as the depth-distance between a reference marker (for example, the reference frames) and the user's eyes can affect the measured geometry and subsequent calculations. Methods including LIDAR, RADAR, SONAR, Structured Light and Photogrammetry can determine the position of physical features in 3D space, rather than 2D space. This be used to create a mesh of three-dimensional points from which specific facial features can be measured and the dimensions of the mesh calibrated against objects or markers of known size and relative positioning (for example, the reference frames or credit card, etc.).

Using LIDAR, RADAR and/or SONAR

Light Detection and Ranging, or Laser Imaging, Detection and Ranging (LIDAR), Radio Detection and Ranging (RADAR), and Sound Navigation and Ranging (SONAR) all measure the time it takes for a signal (light, radio and sound, respectively) to be reflected and returned from a target object. If the position and the direction of the emitted signal is known, using this and the time taken for the reflected signal to return, the distance to the target can be determined. This can be built up over an area by moving the emitter in an array pattern. In this manner ‘point cloud data’ can be collected, which is a set of data points in a 3D coordinate system which can be used to create a 3D ‘mesh’ of the user's face. Using objects of known dimensions, such as the reference frames or credit card as described above, the mesh can be calibrated to determine the real dimensions. This can then be used to determine particular dimensions of the user's face.

An exemplary workflow for using LIDAR, RADAR or SONAR may be as follows:
A user stands still;
The user wears or otherwise presents an object of known size (a reference object, as described above) near to the areas to be measured;
A 3D scanner moves in front of and around the user's face capturing point cloud data;
Distances of the known object are measured on the 3D mesh and used to calibrate the dimensions of the mesh to real life;
Distances on the user's face, such as eye position, are measured on the mesh and adjusted using the calibration factors.

Using this technique, only a single colour mesh is generated, so a physical reference position is preferably included to be visible in the scanned mesh for measurement.

### Using Structured Light

‘Structured light’ scanning projects a known geometry of light, for example one or more straight lines, a grid, or a circle, etc., onto the measured surface. A camera or other vision system can then record an image showing the geometry of the projected light and interpret this into 3D geometry. In particular, the deformation of the known structures of light can be analysed to determine depth and surface topology information.

Again, using this technique, only a single colour mesh is generated, so a physical reference position is preferably included to be visible in the scanned mesh for measurement.

### Photogrammetry

Photogrammetry uses algorithms to identify key features on a series of photographs of the same subject. The relative coordinates of these key features in the 2D images can be mathematically computed to calculate the position of the points in 3D space, to create 3D meshes. Photogrammetry offers the advantage that only readily available hardware is required, and no specialist equipment is required (for example, specialized emitters and receivers).

Furthermore, the generated mesh can be overlaid with the photographic data, creating a coloured 3D mesh. This can advantageously enable specifically coloured surfaces, details and marks such as pupils, irises and reference marks to be accurately identified.

An exemplary workflow for a Photogrammetry is as follows:
A user stands still in an environment with fixed lighting;
The user wears or otherwise presents an object of known size (i.e. a calibration object) near to the areas to be measured;
A camera moves in front of the user's face capturing a series of still images and/or a video;
The series of images and/or video is assessed with photogrammetry software, thereby creating a 3D mesh;
Distances of the known object are measured on the 3D mesh and used to calibrate the dimensions of the mesh to real life;
Distances on the user's face, such as eye position, are measured on the mesh and adjusted using the calibration factors.

Known marks on the calibration object can be used to automatically orientate and size the mesh.

The measured objects are preferably in a static lighting environment for Photogrammetry analysis as changes to the shadows on the object can alter how the key features are identified and calculated. The user to be measured also preferably stays still while all of the photographs and/or video images are captured.

For each of the depth analysis methods described above, a series of images of the user's face viewed from different angles are captured. This can be achieved by a camera moving around a user's face, while the user keeps their head and gaze still. This may be implemented by a user moving the camera themselves, for example a phone camera; or a further person or machinery can rotate the camera around the user's face. In some implementations, a series of cameras may be arranged around the user's face to each take an image of the user's face from a different angle, for example a series of cameras may be arranged in an arc. Alternatively, the user can move their head relative to a static camera, for example, they can rotate their head (i.e. adjusting the jaw). A user should keep their eyes still relative to their face while they do this.

### Manufacture of Bespoke Loupes

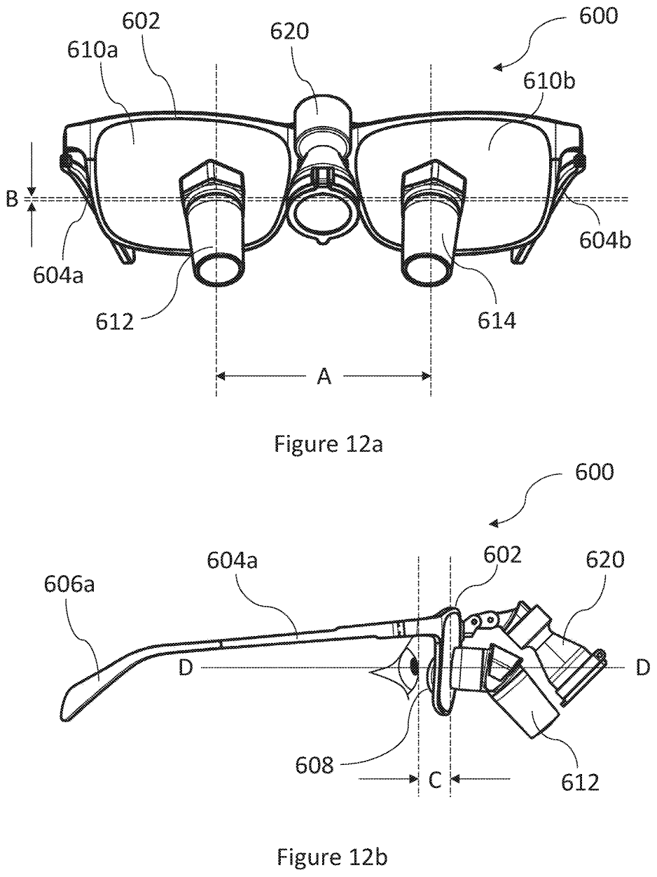

FIGS. 12a and 12b show exemplary magnification loupes 600. The loupes 600 comprise a frame 602 which rests on a user's nose via nose pads 608. The frame 602 is further connected to arms 604a, 604b including temple pads 606a, 606b, which are configured to rest on a wearer's ears. The frame 602 carries lenses 610a, 610b, on each of which is provided a telescope 612, 614 (respectively) configured to perform magnification of the working area (at a working distance). In the illustrated example, the loupes 600 also carry a headlight 620, for illuminating the working area. The headlight 620 is an optional feature.

As discussed above, the measuring tool of the present invention determines values of: interpupillary distance (IPD), individual monocular pupillary distances (monocular PDs); relative pupillary height (PH); and vertex. This is in order to facilitate the manufacture of loupes bespoke to the particular wearer.

Firstly, the IPD is used to determine the width of the frames 602 used, as a correlation between IPD and head width has been established. Frames 602 come in a range of sizes (for example, extra small, small, medium and large), and the size is chosen depending on the appropriate range within which the determined user's IPD falls. For example, if below a first threshold value, extra small frames are used; if between the first threshold value and a second threshold value, small frames are used, and so on.

The IPD, individual monocular pupillary distances (i.e. distance from a defined point to each individual eye pupil centre), and relative PH are used to determine the position of the first telescope 612 within the first lens 610a and the position of the second telescope 614 within the second lens 610b. This is to ensure that the apertures of the telescopes are perfectly aligned with the pupils of the user's eyes. The IPD can be used to define the horizontal distance between the aperture of the first telescope 612 and the aperture of the second telescope 614. This IPD distance is indicated as ‘A’ on FIG. 12a. The relative PH defines the relative heights of the aperture of the first telescope 612 to the aperture of the second telescope 614 relative to a horizontal line. This relative PH distance is indicated by ‘B’ on FIG. 12a. (Note that the relative height of the telescopes is the important parameter, as the positioning of the loupes 600 as a whole relative to the user's face can be tweaked up and down by adjusting the nose pads 608.) Preferably, the determined individual monocular pupillary distances (i.e. distances from a defined point, such as a marker on reference frames and/or the nose bridge, to each individual eye pupil centre) are also used to define the precise positions of the first telescope 612 within the first lens 610a and the position of the second telescope 614 within the second lens 610b.

Finally, the vertex defines the distance between the user's eyelid and the aperture of the telescope 612, 614. The vertex distance is shown as ‘C’ in FIG. 12b (and lies along line D-D). This is used to define the focal distance of the loupes.

In some implementations, the magnification lenses may be provided which account for the eyesight prescription of the user (which typically may optionally be provided by the user). The user can also input their working distance, having determined it themselves. In order to verify the working distance, the user is requested to also input their height. The system calculates an expected range of working distances for that height. If the working distance is not within the expected range, the system may output a prompt for the user to check that the working distance is correct.

### Exemplary User Workflow

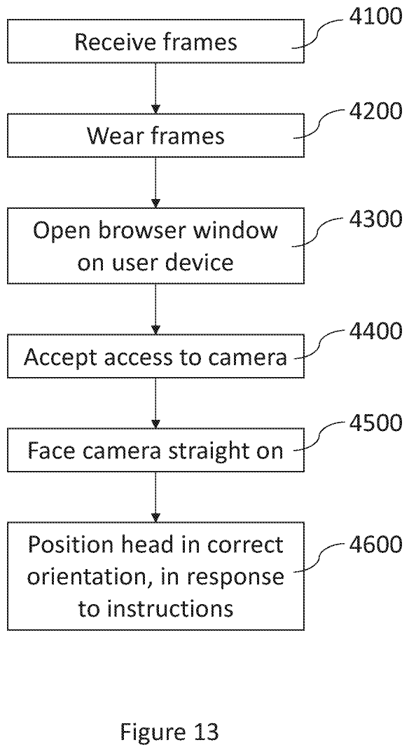

FIG. 13 shows an exemplary workflow of a user interaction with the measurement tool via a web browser, in particular for the situation in which reference frames 500 are used. As a first step, the user receives the reference frames 4100. These may, for example, be received by post. The user then puts the frames 500 on (mounts the frames 500 on their face) so that they are wearing them 4200 and opens the measurement tool in a browser window 4300 on their computer device 200 (these two steps could also be performed in the opposite order). Typically, the user will need to grant permission for the measurement tool to access the camera 212 of their user device 200 at step 4400. The measurement tool now displays a live camera feed 300 on the browser window. At step 4500, the user faces the camera 212 front on, looking straight ahead. The browser window displayed on the user interface 210 typically comprises a dot (or other marker) on the screen, on which the user can focus. This can help in assisting the user to stay focused and continue to look straight ahead at the camera 212. The camera feed 300 displayed on the browser window of the user interface 210 should now be showing an image of the user's face 402. The measuring tool outputs instructions to the user how to adjust their head so that it is at the correct orientation for obtaining the required measurements, and the user positions their head accordingly 4600. This may comprise, for example, moving closer to the camera and tilting their head upwards, upon instructions to do so. (An exemplary orientation process workflow implemented by the measuring tool is shown in FIG. 9.) Once the user's head is at the correct orientation, the measurement tool automatically captures the image.

Alternative user workflows may not utilize the reference frames 500. For example, the first two steps of FIG. 13 may be replaced by a user locating a credit card, which they may hold up to their forehead while facing the camera 4500 and adjusting their orientation 4600.

In a further alternative user workflow, no reference object may be needed (for example, if LIDAR technology is employed). In such a workflow, the user only needs to follow steps 4300 to 4600.

### Alternatives and Possible Modifications

The acceptable threshold values indicated for the orientation process are only exemplary. Alternative values may be defined.

The system and method described above refers to an orienting process and capturing an image via a web browser, the image from which then being processed to calibrate and determine required physical dimensions. In alternative implementations, a user can take a photo, and then upload this photo to the system server 100. In such an implementation, the same image processing is performed to determine the landmark points of the image, and then the orientation processes are performed to determine whether the photo is of sufficiently good quality (i.e. as a validation process). The orientation processes are the same as those described above, for example calculating parameters from the landmark points and determining whether these parameters are within acceptable tolerance ranges. If all the parameters are within the defined acceptable ranges, the image is acceptable and can be used for calibration and determination of the relevant physical dimensions. However, if any of the parameters are determined to be outside the acceptable tolerance range, as the imaging is not performed ‘live’, the system cannot output live feedback to instruct a user how to reorient their head. Instead, a single output can be sent to the user to indicate that the photo is not acceptable. Preferably this output additionally also outputs the reason (for example ‘user head is too distant’ or ‘head tilted to the left’).

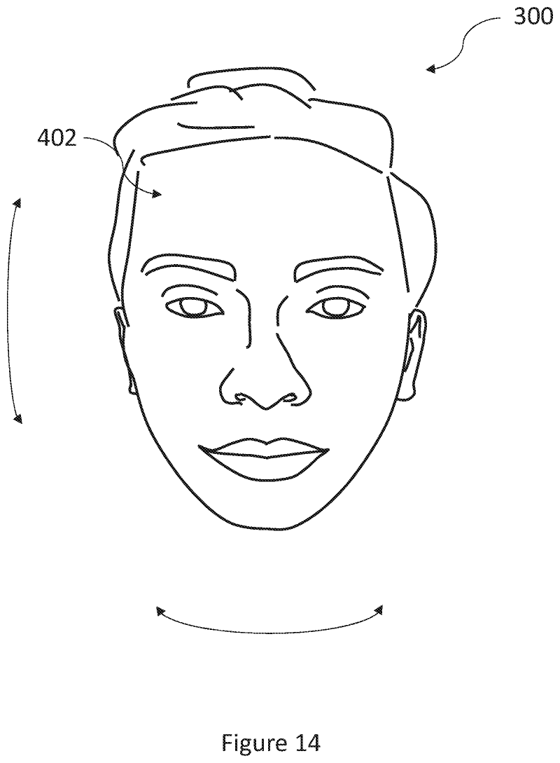

In some further alternative embodiments, a video is recorded of the user moving their head in a set pattern, as directed. A schematic illustration of such a movement is shown in FIG. 14, which shows the user's face 402 within the camera feed 300. For example, the user is instructed to move their head side to side (looking towards their left shoulder and then their right shoulder, or vice versa), thereby adjusting the yaw. They are then instructed to look up and then look down, thereby adjusting the pitch. The calculations of the orientation process, as have been previously described, are applied to the frames of the videos and the parameter values determined for each. The parameter values are tracked over time (for example on parameter value plots over time) and can be mapped to the corresponding image frames. In this manner, the image frames with optimum parameter values can be located from the parameter tracking. These optimum frames can then be extracted and used for calibration and measurement determination. This can avoid the necessity to iteratively and interactively move the user's head, and thereby provide a more streamlined user experience.

These processes for obtaining images can be used in complement to one another, for example to improve confidence in the measurements obtained. Different techniques or even iterations may be used to perform multiple calculations of the required measurements. Statistical analysis can then be performed on the calculated measurements. For example, a mean value can be used. As a further example, it may be defined that the values can only be used if the standard deviation is within a defined threshold; otherwise, the user may be instructed to perform the imaging process (or processes) again.

Alternative animations may be provided in the user display. In some embodiments, the animated overlay displayed to the user may comprise a visualization of what the final loupes will look like, placed in the correct position on the user's face.

In some alternative embodiments, a system may be provided which is a closed system facilitating a wholly autonomous method for measuring for and manufacturing loupes. For example, the system server 100 may be in communication with a manufacture system or device. Measurements can be made in real time with live validation, the algorithm determines frame size from IPD, and working distance from height and gender, thus forming an entirely autonomous measurement system which takes measurements and puts loupes into manufacture. Once the user has interacted with the measurement tool, and the required measurements have been obtained, the server can then automatically send instructions to the manufacture system to manufacture the bespoke loupes using the determined measurements. Such an autonomous system may include one or more checks (for example verification checks) to improve confidence that the measurement values are precise and accurate. For example, the live video orientation process may be used and, in addition, the user is requested to record a video moving their head in a directed manner (as described above; for example, in the manner illustrated in FIG. 14).

In some embodiments, the landmark points can be processed to determine the location of further facial landmarks and dimensions. By way of example, a user's nose size and position, and ear size and position may be determined relative to eye pupil locations. Such a system can use the morphology map of the user's face to further tailor the shape and size of the loupes, for example, type of nose pads, height of the bridge, length of the frame, and/or length of the arms.

It should be understood that the present invention has been described above purely by way of example, and modifications of detail can be made within the scope of the invention.

Each feature disclosed in the description, and (where appropriate) the claims and drawings may be provided independently or in any appropriate combination.

Reference numerals appearing in the claims are by way of illustration only and shall have no limiting effect on the scope of the claims.

1. A method of measuring facial dimensions for manufacturing bespoke loupes, comprising:
receiving live video images of a user's face;
performing validation of the live video images by assessing the orientation of the user's face within the images;
outputting instructions to the user as to how to reorient their head and/or a camera for capturing the images in dependence on the assessed orientation;
capturing at least one image in dependence on the orientation of the user's face; and
determining at least one eye pupil center location within the at least one captured image, defining a datum point within the at least one captured image, and determining the distance between the at least one eye pupil center location and the datum point, wherein said distance defines a monocular pupillary distance.

2. The method of claim 1, wherein the capturing at least one image in dependence on the orientation of the user's face comprises capturing a view of the user's face.

3. (canceled)

4. (canceled)

5. (canceled)

6. (canceled)

7. (canceled)

8. The method of claim 1, wherein the assessing the orientation of the user's face is based on locations of facial landmarks.

9. (canceled)

10. (canceled)

11. (canceled)

12. The method of claim 1, further comprising determining facial landmarks using facial mapping techniques.

13. The method of claim 1, wherein the determining at least one eye pupil centre location comprises interpolating the locations of facial landmarks defined around an eye of a user, preferably around a pupil of the eye, and/or around an iris of the eye.

14. (canceled)

15. (canceled)

16. The method of claim 1, comprising determining the eye pupil centre location for a first eye and a second eye, and determining an interpupillary distance (IPD) as the distance between the eye pupil centre location of the first eye and the eye pupil centre location of the second eye.

17. The method of claim 1, comprising determining the eye pupil centre location for a first eye and a second eye, and determining a relative pupillary height (PH) as the relative height of the eye pupil centre location of the first eye and the eye pupil centre location of the second eye.

18. (canceled)

19. (canceled)

20. (canceled)

21. (canceled)

22. The method of claim 1, wherein the assessing the orientation of a user's face comprises determining at least one nose to cheek angle, wherein the nose to cheek angle is the angle between a nose to cheek line and the horizontal.

23. (canceled)

24. The method of claim 1, wherein the validation comprises determining distance from the camera using focal length of the camera and at least one image of at least one known dimension, preferably wherein the known dimension comprises at least one distance between reference markers and/or an iris diameter.

25. (canceled)

26. (canceled)

27. (canceled)

28. (canceled)

29. (canceled)

30. (canceled)

31. (canceled)

32. (canceled)

33. (canceled)

34. The method of claim 12, further comprising smoothing the positions of the facial landmarks, preferably wherein the smoothing comprises determining average positions over time and/or across a series of images, more preferably normalizing positions over time and/or across a series of images.

35. (canceled)

36. (canceled)

37. (canceled)

38. The method of claim 1, further comprising outputting to a display a marker as a focus point for a user.

39. (canceled)

40. (canceled)

41. (canceled)

42. The method of claim 1, further comprising calibrating dimensions of the image to physical dimensions by reference to a pair of reference frames, wherein the reference frames comprise at least one identifying feature at a known location.

43. (canceled)

44. (canceled)

45. (canceled)

46. (canceled)

47. (canceled)

48. The method of claim 1, further comprising calibrating the image by performing depth analysis using at least one of: LIDAR technology; RADAR technology; SONAR technology; Structured light display, imaging and analysis; and Photogrammetry analysis.

49. The method of claim 48, further comprising determining point cloud data to determine a 3D mesh.

50. The method of claim 1, further comprising capturing as video images a series of images from different positions relative to a user's head.

51. (canceled)

52. The method of claim 1, further comprising manufacturing bespoke loupes, using the facial dimensions.

53. The method of claim 1, wherein the facial dimensions comprise at least one of: interpupillary distance (IPD), monocular pupillary distance, relative pupillary height, and vertex.

54. (canceled)

55. The method of claim 52, wherein a position of telescopes on lenses is determined in dependence on at least one of, and preferably at least two of: interpupillary distance (IPD), monocular pupillary distance, and relative pupillary height.

56. (canceled)

57. A computer program product comprising computer implementable instructions for causing a programmable computer device to carry out the method of claim 1.

58. A tool for measuring facial dimensions for the manufacture of bespoke loupes, wherein the tool comprises computer implementable instructions and at least one programmable computer device, and is configured for receiving live video images of a user's face, and is configured to;
perform validation of the live video images by assessing the orientation of the user's face within the image;
output instructions to the user as to how to reorient their head and/or a camera for capturing the images inn dependence on the assesses orientation;
capture at least one image in dependence on the orientation of the user's face;
determine at least one eye pupil centre location in the at least one captured image; and
define a datum point in the at least one captured image, and determine the distance between the at least one eye pupil centre location and the datum point, wherein said distance defines a monocular pupillary distance.

59. (canceled)

60. (canceled)

61. (canceled)

62. (canceled)

63. (canceled)

64. (canceled)

65. (canceled)

66. (canceled)

67. (canceled)

68. (canceled)

69. (canceled)
